(19) United States
(12) Patent Application Publication
(10) Pub. No.: US 2026/0144852 A1
(43) Pub. Date: May 28, 2026

(54) COMPOSITIONS AND METHODS FOR TREATING ACROMEGALY

(71) Applicant: CAMURUS AB, Lund (SE)

(72) Inventor: Fredrik TIBERG, Lund (SE)

(21) Appl. No.: 19/427,284

(22) Filed: Dec. 19, 2025

Related U.S. Application Data
(63) Continuation of application No. PCT/SE2024/050592, filed on Jun. 18, 2024.

(30) Foreign Application Priority Data
Jun. 20, 2023 (SE) 23302888
Jul. 17, 2023 (SE) 23303290

(51) Int. Cl. A61K 38/31; A61K 9/00; A61K 47/12; A61P 5/06

(52) U.S. Cl. CPC A61K 38/31; A61K 9/0019; A61K 47/12; A61P 5/06

(57) ABSTRACT

This disclosure provides compositions and methods for treating acromegaly.

### CROSS-REFERENCE TO RELATED APPLICATIONS

This application is a continuation of PCT/SE2024/050592, filed on Jun. 18, 2024, which claims priority to Swedish Application No. SE23302888, filed on Jun. 20, 2023, and Swedish Application No. SE23303290, filed on Jul. 17, 2023; the disclosures of which are all incorporated herein by reference.

### BACKGROUND

Acromegaly is a rare, slowly progressing and debilitating disease caused by chronic hypersecretion of growth hormone (GH) by pituitary adenoma that in more than 90% of the cases are benign. The levels of insulin-like growth factor-1 (IGF-1), which mediates most of the growth-promoting actions of GH, are also elevated.

The clinical manifestations of acromegaly are due to the peripheral actions of GH and IGF-1 and local tumor mass effect. The chronic excess of GH and IGF-1 leads to progressive somatic disfigurement due to excessive skeletal growth and soft tissue enlargement, resulting in widening of hands and feet and a characteristic facial aspect, bone deformation and thickening of the skin. Metabolic complications include increased blood glucose levels, hyperinsulinemia, diabetes and dyslipidemia. Respiratory complications can appear as consequences of anatomical changes affecting craniofacial bone and soft tissues (macroglossia and narrowing of upper airways) and alterations in the activity of respiratory muscles. Sleep apnea has been reported in over 60% of patients. Arterial hypertension, arrhythmias and valvular heart disease are also frequently observed in association with acromegaly. Moreover, concentric biventricular hypertrophy is the key feature of cardiomyopathy in patients with acromegaly. Local tumor effects may lead to visual field defects and to hydrocephalus or focal epilepsy in the case of large extensive tumors. Acromegaly can cause a variety of other symptoms, such as headache, excessive sweating, arthralgia and paresthesia. It is well known that patients with acromegaly report impaired health-related quality of life, especially during the active phase of the disease. Patients with acromegaly have a shortened life expectancy, with a mortality rate that is approximately twice that of the general population. The excess mortality is primarily a result of cardiovascular diseases, respiratory complications, diabetes mellitus and malignancies. The prevalence of acromegaly in Europe and the U.S. is estimated to be around 8 cases per 100,000 people, meeting the orphan disease prevalence criteria. The annual incidence rates in these regions range between 0.2 and 1.1 cases per 100,000 people.

The goals of treatment of acromegaly are biochemical normalization, reduction of mortality risk, attenuation of symptoms, control of tumor mass and maintenance of pituitary function. Treatment modalities for acromegaly include surgery, radiotherapy, and medical treatment. Currently, the medical treatment options for acromegaly include somatostatin analogues (SSAs), GH antagonists, and dopamine agonists. The currently available medical treatments in acromegaly have been approved based on efficacy assessed as a biochemical response in terms of IGF-1 normalization and/or GH decrease below a pre-defined threshold.

Octreotide was the first SSA approved for the treatment of acromegaly and is considered the gold standard with well-characterized profiles of efficacy and safety over 30 years of clinical use. Octreotide is available as a short-acting SC formulation, Sandostatin® (approved in the U.S. in 1987), for 2- or 3-times daily administration and as a long-acting release (LAR) formulation, Sandostatin® LAR® (micro-encapsulated octreotide acetate; approved in the US in 1995), administered by intramuscularly (IM) injection every 4 weeks. In most patients, the biochemical effects of octreotide on IGF-1 are seen as early as during the first 3 months of treatment. Treatment with octreotide is associated with a decrease in tumor volume and, in contrast to surgical intervention, the success of octreotide therapy does not seem to be influenced by tumor size. Treatment with octreotide is also associated with improvement in arthralgia, hyperhidrosis, soft tissue swelling, and headache.

The approved dose of Sandostatin® LAR® for the treatment of acromegaly is 20 mg/month. The prescribing information in connection with Sandostatin® LAR® include several warnings relating to increased risk of cardiovascular adverse effects, such as bradycardia and arrhythmia; drug-drug interactions; increased risk of cholelithiasis, hypoglycemia, hyperglycemia, etc. Given that there are only a few products available on the market there is still a need for new treatments improving symptom control and/or treatments, particularly since first-line treatment often involved complicated handling and administrations, e.g., reconstitution prior administration, intramuscular or deep subcutaneous (SC) injections, large needles, no self-administration. Additionally, there is a need for treatments which provide benefits to the patient, and/or caregiver, for example by improving quality of life, ease of administration, e.g., self-administration, etc.

### SUMMARY

This disclosure provides, in part, a lipid composition, comprising 10 or 20 mg of octreotide or a pharmaceutically acceptable salt thereof (e.g., chloride salt) for use in treating acromegaly, and wherein the composition is administered to a patient once every 28 days±7 day (monthly).

The disclosure also provides a method of treating acromegaly, comprising administering to a patient in need thereof a lipid composition comprising 10 or 20 mg of octreotide or a pharmaceutically acceptable salt thereof (e.g., chloride salt), wherein the lipid composition is administered to the patient once every 28±7 days (monthly).

Also provided is a pre-filled syringe, comprising a lipid composition comprising 10 or 20 mg of octreotide or a pharmaceutically acceptable salt thereof (e.g., chloride salt) for use in treating acromegaly, and wherein the composition is administered to a patient once every 28 days±7 day (monthly).

Further provided is an autoinjector, comprising a glass compartment containing a lipid composition comprising 10 or 20 mg of octreotide or a pharmaceutically acceptable salt thereof (e.g., chloride salt) for use in treating acromegaly, and wherein the composition is administered to a patient once every 28 days±7 day ((once every 4 weeks+/−1 week)).

### BRIEF DESCRIPTION OF THE DRAWINGS

FIG. 1 illustrates the clinical trial design.

FIG. 2 illustrates the IGF-1 and GH response.

FIG. 3 illustrates the primary endpoint (ITT).

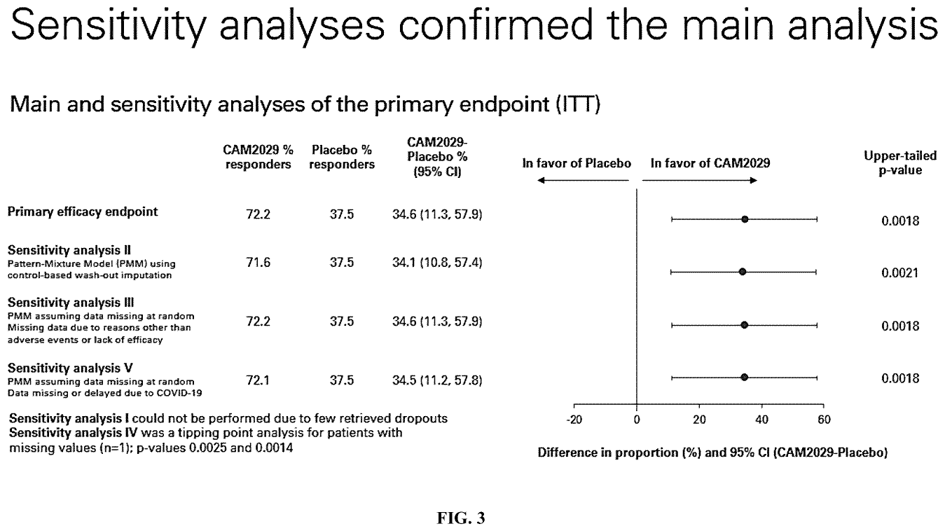

FIG. 4 illustrates supportive analyses of primary endpoint.

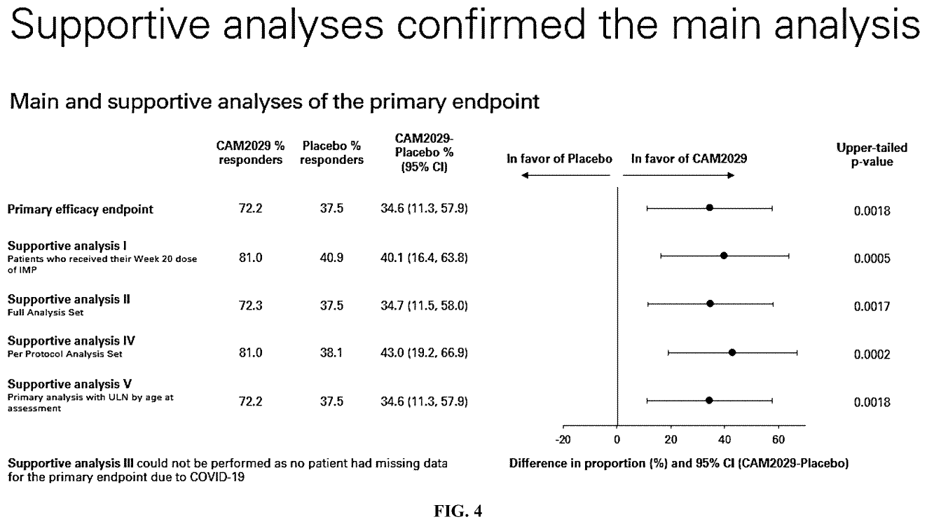

FIG. 5 illustrates the time to loss of response.

FIG. 6 illustrates IGF-1 over time.

FIG. 7 illustrates improvements in quality of life.

FIG. 8 illustrates patient reported satisfaction Scale.

FIG. 9 illustrates an open label treatment.

FIG. 10 illustrates an open label treatment up to 104 weeks.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and understood as by a person of ordinary skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder, and the like.

“Individual,” “patient,” or “subject” are used interchangeably and include any animal, including mammals, preferably mice, rats, other rodents, rabbits, dogs, cats, swine, cattle, sheep, horses, or primates, and most preferably humans. The octreotide and its salts (compound) can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art.

The term “pharmaceutical composition” as used herein refers to a composition comprising at least one compound as disclosed herein formulated together with one or more pharmaceutically acceptable carriers.

In the present specification, the term “therapeutically effective amount” means the amount of the subject compound that will elicit the biological or medical response of a tissue, system, animal or human that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in the treatment of acromegaly.

“Steady state” is the time during which the concentration remains stable or consistent when the drug is given repeatedly. The steady state for the administration of the octreotide compositions disclosed herein is estimated in healthy volunteers to be in the range of 2-3 months, such as about 2 months.

All % are specified by weight herein throughout, unless otherwise indicated. Percent (%) by weight may be abbreviated, e.g., as wt. %. Furthermore, the % by weight indicated is the % of the total lipid composition including all the components indicated herein, unless otherwise indicated.

### Methods

The disclosure provides, in part, a method of treating acromegaly using lipid compositions disclosed herein.

Disclosed herein is a method of treating acromegaly, comprising, consisting essentially of, or consisting of administering to a patient in need thereof a lipid composition comprising 20 mg of octreotide or a pharmaceutically acceptable salt thereof (e.g., chloride salt), wherein the lipid composition is administered to the patient once every 28+/−7 days ((once every 4 weeks+/−1 week), and wherein octreotide is the sole pharmaceutically active ingredient. In an alternative embodiment disclosed herein, a method of treating acromegaly, comprising, consisting essentially of, or consisting of administering to a patient in need thereof a lipid composition comprising 10 mg of octreotide or a pharmaceutically acceptable salt thereof (e.g., chloride salt), wherein the lipid composition is administered to the patient once every 28+/−7 days (monthly, e.g. once every 4 weeks+/−1 week).

### Compounds

The drug product in the present disclosure is octreotide or a pharmaceutically acceptable salt thereof (octreotide chloride, which is considered the same as octreotide hydrochloride). The IUPAC name is (4R,7S,10S,13R,16S,19R)-19-[(2R)-2-amino-3-phenylpropanamido]-10-(4-aminobutyl)-16-benzyl-N-[(2R,3R)-1,3-dihydroxybutan-2-yl]-7-[(1R)-1-hydroxyethyl]-13-[(1H-indol-3-yl)methyl]-6,9,12,15,18-pentaoxo-1,2-dithia-5,8,11,14,17-pentaazacycloicosane-4-carboxamide.

Octreotide has the following chemical structure:

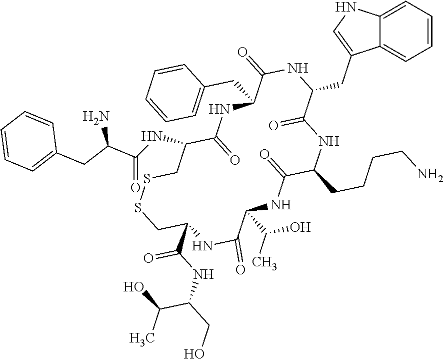 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O]

and the molecular weight is 1019.3 (free peptide, C₄₉H₆₆N₁₀O₁₀S₂).

The amino acid sequence of octreotide is H2N-D-Phe-Cys-Phe-D-Trp-Lys-Thr-Cys-Thr-ol (Disulfide Bridge Cys2-Cys7).

The lipid composition comprising 20 mg/ml octreotide (concentration based on free peptide), is based on glycerol dioleate and phosphatidylcholine as lipid excipients. Additional excipients in the lipid composition may be ethanol (solvent) and propylene glycol (co-solvent). The lipid composition optionally includes EDTA and ethanolamine as additional excipients. The lipid compositions containing octreotide disclosed herein are also referred to as CAM2029. The water content of the lipid composition at release of the product may not be more than (NMT) 1%, determined by USP<921>. Optionally, the lipid composition comprises octreotide chloride. WO2008/152401 (the full disclosure of which is hereby incorporated herein by reference), see, e.g., examples 4-6, discloses a method for obtaining the octreotide chloride and subsequent formulation as a lipid composition. Further examples are disclosed in WO2012/160213 (the full disclosure of which is hereby incorporated herein by reference), see, e.g., examples 1, 2 and 8. The inactive excipients, i.e., the lipids, glycerol dioleate and phosphatidyl choline, the solvent and co-solvent and the optional excipients EDTA and ethanolamine, and the octreotide chloride adds up to at least 80 wt. %, such as 85 wt. % of the total drug product. The rest are essentially by products, for example, originating from the lipids. Glycerol dioleate (GDO) may also contain monoglycerides (NMT 2%) and triglycerides (NMT 5%). Generally, the GDO used in the lipid composition should contain at least about 93% glycerol dioleate. Phosphatidyl choline may also contain lysophosphatidylcholine (NMT 3%) and triglycerides (NMT 2%). Generally, the phosphatidyl choline used in the lipid composition should contain at least about 94% phosphatidyl choline. The composition may consists of 20 mg octreotide base/mL, 6.5% w/w anhydrous ethanol, 6.5% w/w propylene glycol, 0.01% w/w ethylenediaminetetraacetic acid, 0.01% w/w ethanolamine and soybean phosphatidylcholine/glycerol dioleate in a weight ratio 50/50 to final volume.

The lipid formulation may be provided in a pre-filled syringe, e.g., a glass syringe, equipped with a small needle compared to the comparative product (22G). It is not necessary to reconstitute the drug product and, therefore, is considered ready-to-use. Suitable syringes may be those having a volume of about 0.5, about 0.75, about 1.0, about 1.25, and about 1.5 mL. One example is a 1 mL glass syringe, optionally equipped with a small needle compared to the comparative product (22G). Such as 1 mL glass syringe may contain 20 mg octreotide base/mL, 6.5% w/w anhydrous ethanol, 6.5% w/w propylene glycol, 0.01% w/w ethylenediaminetetraacetic acid, 0.01% w/w ethanolamine and soybean phosphatidylcholine/glycerol dioleate in a weight ratio 50/50 to final volume.

Comparative drug product used herein are Sandostatin® LAR® (also referred to as octreotide LAR), which contains octreotide as the acetate salt. It is a long-acting octapeptide with pharmacologic properties mimicking those of the natural hormone somatostatin. Sandostatin LAR® Depot (octreotide acetate for injectable suspension) is available in a vial containing the sterile drug product, which when mixed with diluent, becomes a suspension that is given as a monthly intragluteal injection. The excipients are poly(DL-lactide-co-glycolide) and mannitol (E421) in the vial. Carmellose sodium, mannitol (E421), Poloxamer 188 and water for injections as diluent/solvent. The octreotide is uniformly distributed within the microspheres which are made of a biodegradable glucose star polymer, D,L-lactic and glycolic acids copolymer. Sterile mannitol is added to the microspheres to improve suspendability. Sandostatin LAR may also be referred to as octreotide LAR or Oct LAR in the present disclosure. Sandostatin LAR is provided in a vial and in need of at least 30 min reconditioning before added to a syringe for intramuscular injection using a 20G needle.

SOMATULINE DEPOT (lanreotide) or SOMATULINE Autogel (lanreotide) (ATG) includes lanreotide acetate which is a synthetic cyclical octapeptide analog of the natural hormone, somatostatin. Lanreotide acetate is chemically known as [cyclo S—S]-3-(2-naphthyl)-Dalanyl-L-cysteinyl-L-tyrosyl-D-tryptophyl-L-lysyl-L-valyl-L-cysteinyl-L-threoninamide, acetate salt. Its molecular weight is 1096.34 (base) and its amino acid sequence is:

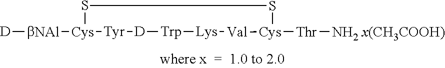

Somatuline DEPOT is a prolonged-release formulation for deep subcutaneous injection. It contains the drug substance lanreotide acetate, water for injection and acetic acid (for pH adjustment). Somatuline ATG also needs to be reconditioned for more than 30 min before subcutaneous injection to a patient via a 18G/19G syringe.

### EXCIPIENTS

The present disclosure also relates to a lipid composition comprising, consisting essentially of, or consisting of 10 or 20 mg of octreotide or a pharmaceutically acceptable salt thereof, and glycerol dioleate (GDO), phosphatidyl choline (PC), ethanol, and optionally propylene glycol (PG). The lipid compositions may be substantially non-aqueous.

The lipid compositions may form a depot composition upon contact with an aqueous fluid. The term “depot” relates to the composition which is formed upon exposure of the lipid composition to excess aqueous fluid, e.g., as occurs during numerous parenteral administration routes, such as administration in the subcutaneous tissue of a human patient. The depot typically has a much higher viscosity than the corresponding lipid composition and provides for the gradual release of the octreotide contained within the depot.

### Glycerol Dioleate (GDO)

The lipid composition contains glycerol dioleate (GDO). The GDO may be present in the lipid composition in an amount ranging from 20 to 90 wt. % of the lipid composition (e.g., 30 to 70 wt. %, 33 to 60 wt. %, 43 to 55 wt. %, 38 to 43 wt. %).

Since GDO may be derived from natural sources, there is generally a certain proportion of “contaminant” lipid having other chain lengths, etc. In this context, “pure” GDO is a di-ester of glycerol and two C18:1 fatty acids. Any other diacyl glycerol is considered to be an impurity. In one aspect, GDO, as used herein, indicates any commercial grade of GDO with concomitant impurities (i.e., GDO of commercial purity). These impurities may be separated and removed by purification but, providing the grade is consistent, this is rarely necessary. If necessary, however, “GDO″” may be essentially chemically pure GDO, such as at least 70% pure GDO (e.g., at least 75% pure, at least 80% pure, at least 85% pure, at least 90% pure, at least 93%, at least 95% pure, at least 98% pure, at least 99% pure (area %)). In some embodiments, the diglycerides content of the glycerol dioleate is about 92%-100%, such as about 93% to about 99%. The GDO used herein should have an oleic acid content (C18:1) of at least 80% (e.g., at least 85%, at least 90%, at least 95%, at least 98%, at least 99%). In some embodiments, the diglyceride content is about 93% to about 99%, and the oleic acid content is about 88% to about 99% (area %).

Any material used in the lipid composition, including GDO, may potentially include unavoidable trace impurities of metals, optionally including heavy metals. A typical maximum concentration of heavy metals (or elemental impurities) in GDO is 5 ppm.

In alternative embodiments, GDO may be replaced by, or combined with, at least one other lipid, e.g., at least one fatty acid and/or fatty acid ester (lipid). Fatty acids/lipids contain a polar carboxylic acid or ester “head group” with the hydrocarbon chain forming a non-polar “tail” group. Fatty acid esters are esterified fatty acids. Fatty acids or esters used in the lipid composition may be solid or liquid at room temperature and pressure. Examples of non-polar “tail” groups include C6-C32 alkyl and alkenyl groups, which are typically present as long chain carboxylic acids or the esters thereof. These are often described by reference to the number of carbon atoms and the number of unsaturations in the carbon chain. Thus, CX: Z indicates a hydrocarbon chain having X carbon atoms and Z unsaturations. Examples particularly include caproyl (C6:0), capryloyl (C8:0), capryloyl (C10:0), lauroyl (C12:0), myristoyl (C14:0), palmitoyl (C16:0), phytanoyl (C16:0), palmitoleoyl (C16:1), stearoyl (C18:0), oleoyl (C18:1), elaidoyl (C18:1), linoleoyl (C18:2), linolenoyl (C18:3), arachidonoyl (C20:4), behenoyl (C22:0), and lignoceroyl (C24:9) groups. When reference is made herein to the number of carbon atoms in the “chain” or “tail” this number includes the carbon atom of the —C(O)O-moiety, as is conventional in the art.

Thus, typical non-polar chains are based on the fatty acids of natural ester lipids, including caproic, caprylic, capric, lauric, myristic, palmitic, phytanic, palmitolic, stearic, oleic, elaidic, linoleic, linolenic, arachidonic, behenic, or lignoceric acids, or the corresponding alcohols.

The lipid(s) may be saturated or unsaturated. For example, the lipid(s) may comprise at least 1 wt. % unsaturated lipid (based on the total lipid content) (e.g., at least 5 wt. % (5-100%), at least 15 wt. % (15-100%), at least 30 wt. % (30-100%), at least 50 wt. % (50-100%), at least 80 wt. % (80-100%)).

GDO may be replaced or combined with, for example, an edible lipid such as almond oil, avocado oil, butter, canola oil, castor oil, coconut oil, corn oil, cottonseed oil, flaxseed oil, ghee, lard, linseed oil, macadamia oil, margarine, mustard oil, olive oil, palm oil, peanut oil, pumpkin seed oil, rice bran oil, safflower oil, sesame oil, soybean oil, sunflower oil, tea seed oil, vegetable oil, or walnut oil.

One particular example is tocopherol, which may be used to replace GDO.

When GDO is replaced by at least one other lipid, the at least one lipid may be present in the lipid composition in an amount ranging from 20 to 90 wt. % of the lipid composition (e.g., 30 to 70 wt. %, 33 to 60 wt. %, 43 to 55 wt. %, 38 to 43 wt. %). If the lipid composition contains both GDO and at least one additional lipid, the combination may also be present in the lipid composition in an amount ranging from 20 to 90 wt. % of the lipid composition (e.g., 30 to 70 wt. %, 33 to 60 wt. %, 43 to 55 wt. %, 38 to 43 wt. %).

### Phosphatidyl Choline (PC)

The lipid composition also contains phosphatidyl choline (PC). PC is present in an amount ranging from 20 to 80 wt. % of the lipid composition (e.g., 30 to 70 wt. %, 33 to 60 wt. %, 35 to 55 wt. %, 38 to 43 wt. %). Ratios of GDO:PC may be 40:60 to 70:30 (e.g., 45:55 to 55:45, 40:60 to 54:46, 45:55 to 54:46, 47:53 to 53:47). Ratios of around 50:50 (e.g., 49:51 to 51:49) may be highly effective.

In alternative embodiments, PC may be replaced by, or combined with, at least one other phospholipid. The phospholipids may be derived from a natural source. In the case of PC, suitable sources of phospholipids include egg, heart (e.g., bovine), brain, liver (e.g., bovine), and plant sources, including vegetable PC, such as soybean. Such sources may provide one or more constituents of the PC and/or at least one other phospholipid. Any single PC or mixture thereof may be used according to the present disclosure, although egg, heart (e.g., bovine), brain, liver (e.g., bovine), and plant sources, including vegetable PC, such as soybean or any mixture thereof are used in most embodiments disclosed herein.

The PC may be derived from soy. The PC may comprise 18:2 fatty acids as the primary fatty acid component with 16:0 and/or 18:1 as the secondary fatty acid components. These may be present in the PC at a ratio of between 1.5:1 and 6:1. PC having approximately 60-65% 18:2, 10 to 20% 16:0, 5-15% 18:1, with the balance predominantly other 16 carbon and 18 carbon fatty acids may be used, e.g., soy PC. In some embodiments, the PC has a purity of not less than 90%, not less than 92%, not less than 94%, such as about 94%-100% (based on dry weight). The maximum lysophosphatidylcholine content is about 3%, e.g., not more than 3%. The maximum triglycerides content is about 2%, such as not more than 2%. In some embodiments, the PC used in the lipid composition has a purity of about 94%-100% (based on dry weight), and contains not more than 3% lysophosphatidylcholine and optionally not more than 2% triglycerides.

Alternatively, the PC component may comprise synthetic dioleoyl PC (DOPC). The use of DOPC may provide increased stability and so may be used for compositions needing to be stable to long term storage, and/or having a long release period in vivo. Here, the PC component may contain at least 50% synthetic dioleoyl PC (e.g., at least 75% synthetic dioleoyl PC) and even essentially pure synthetic dioleoyl PC. Any remaining PC can be of any sort. DOPC may be more expensive.

Alternative types of phospholipids are synthetic or highly purified PCs, such as dioleoyl phosphatidyl choline (DOPC), may, in alternative embodiments, be used as all or part of the phospholipid component. The synthetic dioleoyl PC may be 1,2-dioleoyl-sn-glycero-3-phosphocholine, and other synthetic PC components include DDPC (1,2-Didecanoyl-sn-glycero-3-phosphocholine); DEPC (1,2-Dierucoyl-sn-glycero-3-phosphocholine); DLOPC (1,2-Dilinoleoyl-sn-glycero-3-phosphocholine); DLPC (1,2-Dilauroyl-sn-glycero-3-phosphocholine); DMPC (1,2-Dimyristoyl-sn-glycero-3-phosphocholine); DOPC (1,2-Dioleoyl-sn-glycero-3-phosphocholine); DPPC (1,2-Dipalmitoyl-sn-glycero-3-phosphocholine); DSPC (1,2-Distearoyl-sn-glycero-3-phosphocholine); MPPC (1-Myristoyl-2-palmitoyl-sn-glycerol-3-phosphocholine); MSPC (1-Myristoyl-2-stearoyl-sn-glycero-3-phosphocholine); PMPC (1-Palmitoyl-2-myristoyl-sn-glycero-3-phosphocholine); POPC (1-Palmitoyl-2-oleoyl-sn-glycero-3-phosphocholine); PSPC (1-Palmitoyl-2-stearoyl-sn-glycero-3-phosphocholine); SMPC (1-Stearoyl-2-myristoyl-sn-glycero-3-phosphocholine); SOPC (1-Stearoyl-2-oleoyl-sn-glycero-3-phosphocholine); and SPPC (1-Stearoyl-2-palmitoyl-sn-glycero-3-phosphocholine), or any combination thereof.

Alternative lipid compositions that may be used in the present disclosure are disclosed in WO2016/066655, which is incorporated herein by reference, where lipid slow-release matrices based on triacyl lipids that can form depot compositions on exposure to aqueous fluids without the need for a phospholipid component to be present, though in certain embodiments a phospholipid may also be present.

Alternative embodiments include phosphatidylethanolamine (PE), phosphatidylserine, and phosphatidylinositol, instead of phosphatidyl choline.

Since the lipid compositions are to be administered to a subject, such as a patient, with the inclusion of octreotide, the components should be biocompatible. In this regard, PC (such as soy PC and/or DOPC) and GDO are useful, since they are well tolerated and are broken down in vivo into components that are naturally present in the mammalian body. Appropriate amounts of each component suitable for the combination are those amounts indicated herein for the individual components in any combination. This applies also to any combinations of components indicated herein, where context allows.

When PC is replaced by at least one other phospholipid, the at least one phospholipid may be present in the lipid composition in an amount ranging from 20 to 80 wt. % of the lipid composition (e.g., 30 to 70 wt. %, 33 to 60 wt. %, 35 to 55 wt. %, 38 to 43 wt. %). If the lipid composition contains both PC and at least one additional phospholipid, the combination may also be present in the lipid composition in an amount ranging from 20 to 80 wt. % of the lipid composition (e.g., 30 to 70 wt. %, 33 to 60 wt. %, 35 to 55 wt. %, 38 to 43 wt. %). Ratios of GDO and/or the at least one lipid other than GDO:PC and/or the at least one phospholipid other than PC may be 40:60 to 70:30 (e.g., 45:55 to 55:45, 40:60 to 54:46, 45:55 to 54:46, 47:53 to 53:47). Ratios of around 50:50 (e.g., 49:51 to 51:49) may be highly effective.

### Solvent

The lipid composition also contains the solvent ethanol. In alternative embodiments, the ethanol may be replaced by, or combined with, at least one other biocompatible organic solvent. Since the lipid composition may generate a depot composition following administration (e.g., in vivo), typically upon contact with excess aqueous fluid, it is desirable that this solvent be tolerable to the subject and be capable of mixing with the aqueous fluid, e.g., body fluids, and/or diffusing or dissolving out of the lipid composition into the aqueous fluid. Therefore, solvents having at least moderate water solubility may be used. In some embodiments the lipid compositions include a polar co-solvent.

In alternative embodiments, instead of (or in addition to) ethanol, the solvent in the lipid composition comprises, consists essentially of, or consists of at least one solvent selected from the group consisting of: alcohols, amines, amides, and esters. Therefore, the solvent may comprise at least a mono-alcoholic solvent, such as ethanol, propanol, iso-propanol, or mixtures thereof. In one embodiment, the solvent is ethanol. The lipid composition may also comprise, consist essentially of, or consist of a mono-alcoholic solvent (e.g., ethanol) and a polar co-solvent (e.g., propylene glycol). The amount of solvent in the lipid composition may affect several features, including, for example, the viscosity and the rate (and duration) of release. Therefore, the amount of solvent may be at least sufficient to provide a low viscosity mixture but may additionally be determined so as to provide the desired release rate. Typically, a level of 1 to 30% (e.g., 2 to 20%, 3 to 18%, 4 to 16%, 5 to 15%) solvent will provide suitable release and viscosity properties. In some embodiments, the solvent (e.g., EtOH) is at a level of 3-10% (e.g., 4-9%, 5-8%, 6-7%), and a co-solvent (e.g., PG) is at a level of 3-10% (e.g., 4-9%, 5-8%, 6-7%).

As indicated above, the amount of solvent in the lipid compositions may be at least sufficient to provide a low viscosity mixture (e.g., a molecular solution) of the components of the lipid composition and can be determined for any particular combination of components by standard methods.

The solvent may be a single solvent (e.g., ethanol) or a mixture of suitable solvents (e.g., ethanol and PG) but will generally be of low viscosity. In some embodiments the solvent is a mixture of ethanol and propylene glycol, and no other solvent. The viscosity of the “low viscosity” solvent (single solvent or mixture) may be no more than 18 mPas at 20° C. (e.g., no more than 15 mPas at 20° C., no more than 10 mPas at 20° C., no more than 7 mPas at 20° C.).

WO2012/160213 describes the addition of a polar solvent in addition to a mono-alcoholic solvent results in numerous advantages including reduced viscosity and reduced active agent burst profile. In addition to the aspects described previously for the solvent component, In some embodiments, the solvent comprises a mono-alcoholic solvent (e.g., ethanol) and a polar co-solvent. The term “polar co-solvent” as used herein defines a solvent having a dielectric constant (diel) of at least 28 at 25° C. (e.g., at least 30 at 25° C.) but is not water or any aqueous fluid. Examples include propylene glycol (diel-32), and N-methyl-2-pyrrolidone (NMP, diel-32). The levels of solvent recited herein may apply equally to mixtures of mono-alcoholic solvent and a polar co-solvent unless context permits otherwise.

In another embodiment, the solvent comprises, consists essentially of, or consists of a mixture of a mono-alcoholic solvent and a polar co-solvent. The polar co-solvent may be a di-alcoholic C3-C6 organic solvent, i.e., a C3-C6 organic solvent comprising two hydroxy groups. The di-alcoholic solvent may be propylene glycol. When present, a polar co-solvent may be included at a level of 2 to 12 wt. % of the lipid composition (e.g., 3 to 10 wt. %, 4 to 9 wt. %, 5 to 8 wt. %, 6 to 7 wt. %). This level is counted as part of the ranges recited above for the solvent. In one embodiment, the solvent comprises, consists essentially of, or consists of a mixture of ethanol and propylene glycol (PG), where PG is considered a co-solvent.

Where both an organic mono-alcoholic solvent and a polar co-solvent are present, e.g., ethanol and PG, the ratio of mono-alcoholic solvent to polar co-solvent solvent may be in the range 20:80 to 70:30 (w/w) (e.g., 30:70 to 70:30 (w/w), 40:60 to 60:40 (w/w), 45:55 to 55:45 (w/w), about 50:50 (w/w)). The amounts and ratio of ethanol and PG may have an effect on properties such as release of an active agent, viscosity of the lipid composition, etc., features which are all important characteristics of suitable lipid compositions for subcutaneous injection of an active agent to a patient in need thereof.

In one embodiment, the solvent is present at a level of 1 to 30 wt. % (e.g., 5 to 20 wt. %, 8 to 18 wt. %, 10 to 15 wt. %) and comprises, consists essentially of, or consists of a mixture of ethanol and PG, wherein the ratio of ethanol to PG (w/w) is in the range of 30:70 to 70:30 (w/w) (e.g., 40:60 to 60:40 (w/w), 45:55 to 55:45 (w/w), 47:53 to 53:47 (w/w), about 50:50 (w/w)). Additional examples are about 4-10 wt. % ethanol and about 4-10 wt. % PG, e.g., about 5-8 wt. % (e.g., 6-7 wt. %) of each.

Even where a polar co-solvent is present in the lipid composition, the total water level may remain at release of the lipid composition for sale at the levels described herein (e.g., 1.0 wt. % or less, 0.1 to 1.0 wt. %).

The term “release” in this context means released for administration to a patient, i.e., a pharmaceutical product approved by a regulatory agency, e.g., the FDA. The term release specification means the tests and limits against which raw material, intermediate, and final product are measured prior to use and/or release.

### Optional Component—EDTA

The lipid composition may also optionally contain EDTA (“ethylenediamine tetraacetic acid” or “edetic acid”). EDTA may be present in 0.005-0.02 wt. %., such as 0.007-0.015 wt. %, such as about 0.01 wt. %. As used herein, the term “EDTA” may represent ethylenediammetetraacetic acid as such. Alternatively, EDTA as indicated herein may include ethylenediammetetraacetic acid itself, EDTA analogues, and alkylammonium EDTA salts. “EDTA” herein thus includes “EDTA, analogues thereof, and alkylammonium EDTA salts,” whenever context allows. “EDTA” does not include the disodium salt of EDTA.

Examples of EDTA analogues include:
Iminodiacetic acid (IDA)-(NH(CH₂CO₂H)₂;
Nitrilotriacetic acid (NTA)-N(CH₂CO₂H)₃;
Pentetic acid*-N(CH₂CO₂H)₂CH₂CH₂N(CH₂CO₂H)CH₂CH₂N(CH₂CO₂H)₂;
Egtazic acid-N(CH₂CO₂H)₂CH₂CH₂OCH₂CH₂OCH₂CH₂N(CH₂CO₂H)₂;
NOTA-[N(CH₂CO₂H)CH₂CH₂]₃; and
DOTA-[N(CH₂CO₂H)CH₂CH₂]₄.
*Also known as “DTPA”

EDTA analogues and alkylammonium salts thereof are further disclosed and described in WO 2018/060212, which is hereby incorporated herein by reference.

The alkylammonium salt(s) of EDTA is provided by contacting the EDTA or analogue thereof with a suitable alkylamine, examples are:
Ethanolamine “ETA” (NH₂(CH₂CH₂OH));
Diethanolamine “DIETA” (NH(CH₂CH₂OH)₂);
meglumine (NH(CH₃)CH₂(CHOH)₄CH₂OH));
tris-hydroxymethylamine “TRIS” (N(CH₂OH)₃);
ethylenediamine (NH₂CH₂CH₂NH₂); or
serinol (NH₂CH(CH₂OH)₂).

The mass of the alkylammonium cation of Formula (I) may be below 500 amu (e.g., below 350, below 250 amu). Salts of EDTA containing the ethanolammonium ion (HOCH₂CH₂NH₃) may be used. The EDTA salt may be a salt of EDTA with ethanolamine (ETA) (e.g., EDTA with ETA only).

The lipid composition may also contain EDTA salts comprising an anion of EDTA and at least one alkylammonium cation of the suitable alkylamine as previously described.

The provision of EDTA and/or a structural analogue thereof and a suitable alkylamine, e.g., ethylenediamine tetraacetic acid and ethanolamine, or ethylenediamine tetraacetic acid and diethanolamine, is believed to enable the solubilization of EDTA or a structural analogue thereof by forming an alkylammonium salt of EDTA or structural analogues thereof. Contrary to the inorganic sodium salt of EDTA, i.e., EDTA disodium salt, the provision of the alkylamine and the EDTA or structural analogues thereof enables substantial amounts of EDTA in the solution of lipid components. Further details are available in WO 2018/060212.

### Optional Excipients

Besides the octreotide or pharmaceutically acceptable salt thereof, the lipid composition may contain additional excipient(s).

The octreotide dissolved in the lipid composition, may gain stability (both storage and in vivo stability) by certain stabilizing additives. Such additives include, but are not limited to, sugars (e.g., sucrose, trehalose, lactose, etc.), polymers (e.g., polyols such as carboxy methyl cellulose), amino acids (such as methionine, glutamate, lysine, etc.), lipid-soluble acid components such as HCl, anionic lipids, and/or surface active agents (such as dioleoyl phosphatidyl glycerol (DOPG), palmitoyloleoyl phosphatidylglycerol (POPG), and oleic acid (OA)). Although all of the above are possible ways to alter the properties of lipid compositions disclosed herein, the EDTA is generally deemed as sufficient to achieve the necessary stability of the lipid composition.

Single-dose formats, such as pre-filled syringes comprising lipid compositions disclosed herein, must remain stable and potent in storage prior to use but are disposable after the single use. The substantially non-aqueous lipid compositions have enhanced storage stability at elevated temperatures, such as at 25° C. or even 40° C. This offers advantages in terms of ease of transportation and storage (no need for refrigeration). A single dose format of the lipid composition may have stability such that after storage for 2 months at 25° C. (with air in head space), the assayed octreotide concentration is at least 95% that of the initial assayed octreotide concentration, and after 3 months, the assayed octreotide concentration is at least 90% that of the initial assayed octreotide concentration, the lipid composition disclosed herein generally has an acceptable storage stability of twelve months or longer.

A single dose format of the lipid composition may have a stability such that after storage for 2 months at 40° C. (with air in head space), the assayed octreotide concentration is at least 85% that of the initial assayed octreotide concentration, and after 3 months, the assayed octreotide concentration is at least 80% that of the initial assayed octreotide concentration.

Therefore, the lipid compositions can optionally contain an antimicrobial or microbial-static agent, which includes bacteriostatic agents and preservatives. Such agents include benzalkonium chloride, m-cresol, benzyl alcohol, or other phenolic preservatives. Typical concentrations as known in the art can be used. In most aspects and embodiments, there are no antimicrobial or microbial-static agents added.

These additional components, if present, may be present in an amount of 0 to 5 wt. % (e.g., 0.01 to 4 wt. %, 0.1 to 3 wt. %, 1 to 2 wt. %), such as no more than 2% by weight, or no more than 1% by weight.

In some embodiments the lipid composition comprises no additional excipient, only EDTA and an alkylamine.

### Water Content

It is difficult to eliminate all traces of water (especially from the raw materials). Even if essentially water-free formulations could be achieved, lipid compositions described in this disclosure will typically be stored in ready-to-use form, e.g., in syringes and possibly under refrigerated conditions. Syringes are often not completely air-tight meaning that the level of water in the lipid composition may increase to an appreciable level over time, e.g., over months, even if the initial level of water is insignificant.

The initial absolute level of water in the lipid composition may be between 0 to 1.0 wt. % (e.g., less than 1.0 wt. %, less than 0.8 wt. %, less than 0.5 wt. %). For example, the level of water may be in the range of 0.1 to 0.9 wt. %, such as 0.2 to 0.8 wt. %, 0.3 to 0.7 wt. %, or 0.4 to 0.6 wt. %. These levels refer to the absolute level of water and not added levels of water. Any unavoidable trace of water present within the components of the lipid composition is included in this stated level of water. After 3 months of storage, the absolute water level may be no more than 1.5 wt. %. Absolute levels of water can be measured by methods well known in the art, such as Karl Fischer titration. For example, the water content may be measured according to the procedure in United States Pharmacopoeia (USP 40-NF 35, USP <921> Water determination, Method 1a).

The lipid composition may have a water content of between 0 to 1.0 wt. % (e.g., less than 1.0 wt. %, less than 0.8 wt. %, less than 0.5 wt. %) when the lipid composition product is released for sale (e.g., released for administration to a patient).

### Embodiments

The present disclosure relates to a method of treating acromegaly, comprising, consisting essentially of, or consisting of administering to a patient in need thereof a lipid composition comprising 10 or 20 mg of octreotide or a pharmaceutically acceptable salt thereof, wherein the lipid composition is administered to the patient once every month, e.g. once every 4 weeks+/−1 week (28+/−7 days).

The lipid composition, which is described herein, may be administered to the patient once every month as a single unit-dose, or in multi-dose formats.

The lipid composition may be administered to the patient by subcutaneous injection. The lipid composition may be administered to the patient by a syringe, a pre-filled syringe, an autoinjector, or a pen injector.

The lipid composition may be administered to the patient not more than once every month, and in a volume of about 1 mL.

The lipid formulation may be provided in a pre-filled syringe, e.g., a 1 mL glass syringe, equipped with a small needle compared to the comparative product (22G), and the drug product is not necessary to be reconstituted and considered ready-to-use.

The bioavailability of octreotide or a pharmaceutically acceptable salt thereof has been shown to be higher for a lipid composition of the present disclosure administered subcutaneously than for Sandostatin® LAR® which is administered as an intramuscular injection. Completed Phase I clinical trials showed that octreotide release from a lipid composition of the present disclosure had a rapid onset of action, with an octreotide Cₘₐₓ observed within approximately 4 to 24 hours after dosing. See Tiberg et al., British J. Clin. Pharmacol. 80(3):460-472 (2015). Thereafter, the plasma concentration slowly declined over time with therapeutic drug levels maintained for approximately 4 weeks, resulting in observable suppression of insulin-like growth factor-1 over 4 weeks after dosing. Dose-proportional PK was observed in the dose range of 10 to 30 mg of the lipid composition. The bioavailability of octreotide was approximately 5 times higher for a lipid composition of the present disclosure than for Sandostatin® LAR®.

The method of administering the lipid composition of the present disclosure as a small-volume subcutaneous injection in, for example, a pre-filled syringe (i.e., eliminating the need for reconstitution) with, for example, a thin needle may provide a ready-to-use, long-acting octreotide formulation, with a higher octreotide bioavailability than Sandostatin® LAR®, and may lead to improved quality of patient care and treatment convenience.

The present disclosure may not be appropriate for patients with long QT syndrome, a family history of idiopathic sudden death or congenital long QT syndrome, or any of the following: Risk factors for Torsades de Pointes, including uncorrected hypokalemia or hypomagnesemia, history of cardiac failure, or history of clinically significant/symptomatic bradycardia; Treatment with concomitant medication(s) with a “known risk of Torsades de Pointes” per www.qtdrugs.org that cannot be discontinued or replaced with safe alternative medication at least 7 days or 5 half-lives (whichever is longer) before the first administration of the lipid composition of the present disclosure; or a baseline QTc interval corrected by Fridericia's formula (QTcF) of >450 msec for male patients and >470 msec for female patients before the first administration of the lipid composition of the present disclosure.

Upon administration of the lipid composition of the present disclosure to a patient in need thereof, the dosing of the lipid composition may not need to be interrupted for male patients having an average QTcF of 450 msec to 480 msec and for female patients having an average QTcF of 470 msec to 480 msec.

Upon administration of the lipid composition of the present disclosure to a patient in need thereof, for male and female patients having an average QTcF of 481 msec to 500 msec, the dosing of the lipid composition may need to be interrupted/paused/delayed until the average QTcF of the patient falls below 481 msec, whereupon the dosing of the lipid composition may resume.

### EXEMPLARY EMBODIMENTS

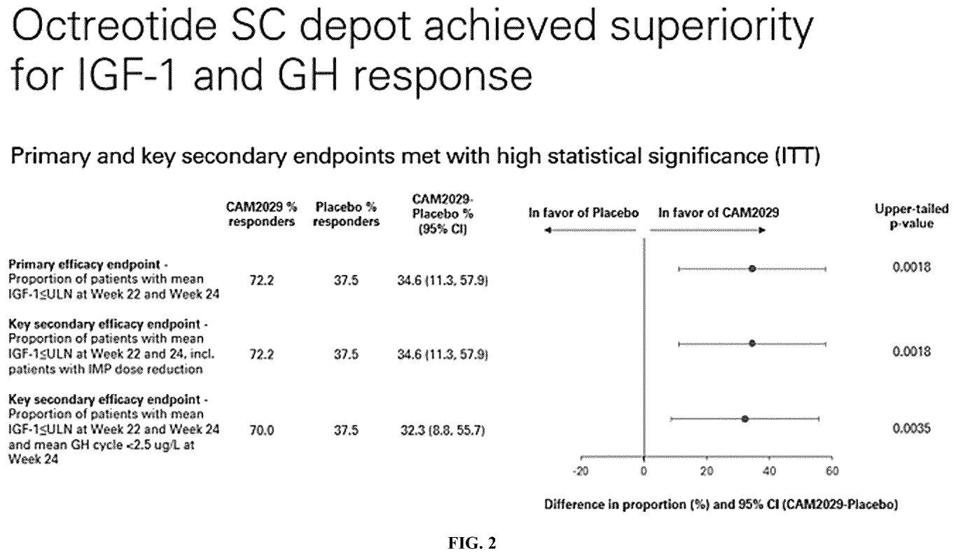

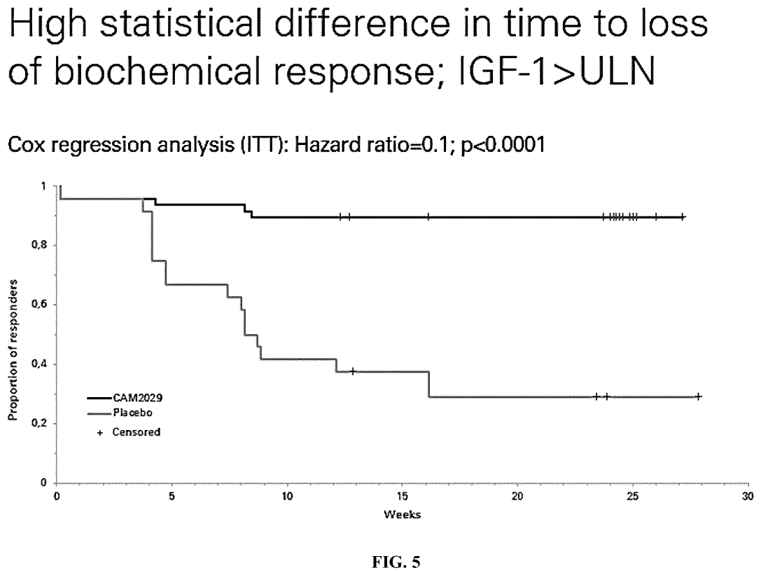

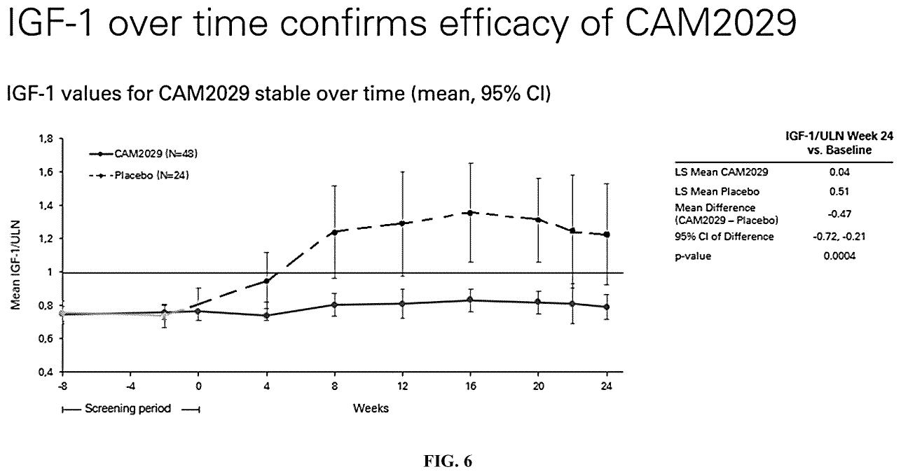

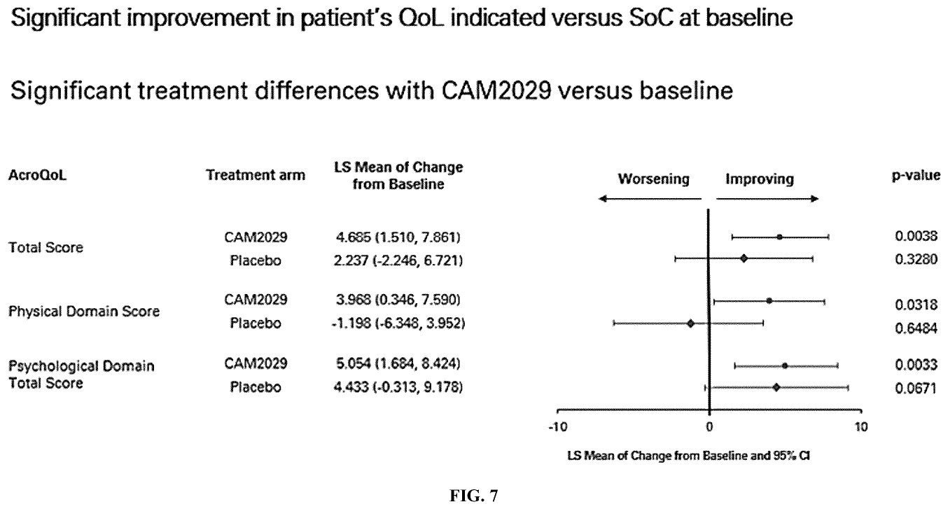

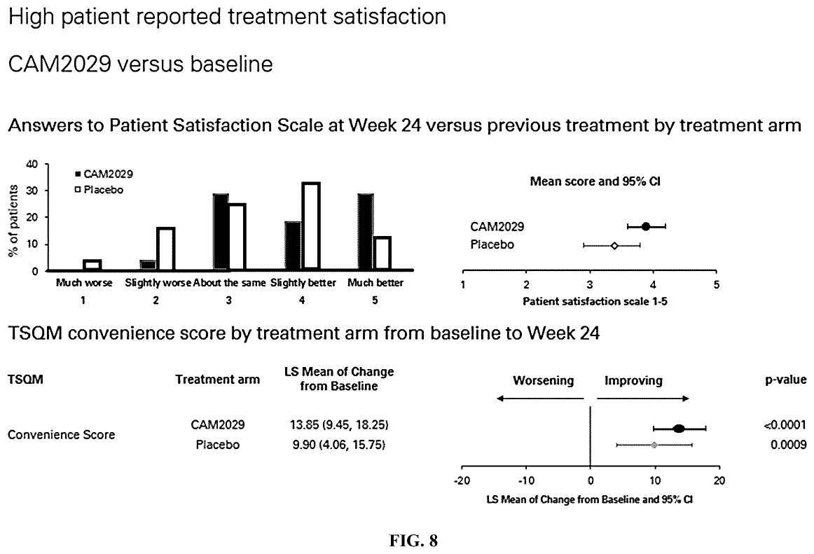

E1. A method of treating acromegaly, comprising, consisting essentially of, or consisting of administering to a patient in need thereof a lipid composition comprising, consisting essentially of, or consisting of 10 or 20 mg of octreotide or a pharmaceutically acceptable salt thereof, wherein the lipid composition is administered to the patient once every 28+/−7 days (once every 4 weeks+/−1 week).
E2. The method of E1, wherein the lipid composition is administered as a unit dose.
E3. The method of E1 or E2, wherein the lipid composition comprises 20 mg octreotide, or an equivalent amount of a pharmaceutically acceptable salt thereof.
E4. The method of any one of E1-E3, wherein the pharmaceutically acceptable salt thereof is hydrochloride (chloride).
E5. The method of E4, wherein the octreotide, or pharmaceutically acceptable salt thereof is the sole active ingredient in the lipid composition.
E6. The method of any one of E1-E5, wherein the lipid composition further comprises, consists essentially of, or consists of glycerol dioleate, phosphatidylcholine, and ethanol.
E7. The method of any one of E1-E6, wherein the lipid composition comprises, consists essentially of, or consists of the octreotide or pharmaceutically acceptable salt thereof (e.g., octreotide chloride), glycerol dioleate, phosphatidylcholine, ethanol, and propylene glycol.
E8. The method of any one of E1-E7, wherein the lipid composition comprises, consists essentially of, or consists of the octreotide or pharmaceutically acceptable salt thereof (e.g., octreotide chloride), glycerol dioleate, phosphatidylcholine, ethanol, and EDTA.
E9. The method of any one of E1-E8, wherein the lipid composition comprises, consists essentially of, or consists of the octreotide or pharmaceutically acceptable salt thereof (e.g., octreotide chloride), glycerol dioleate, phosphatidylcholine, ethanol, propylene glycol, and EDTA.
E10. The method of any one of E1-E9, wherein the lipid composition comprises, consists essentially of, or consists of the octreotide or pharmaceutically acceptable salt thereof (e.g., octreotide chloride), glycerol dioleate, phosphatidylcholine, ethanol, propylene glycol, EDTA, ethanolamine and/or diethanolamine.
E11. The method of any one of E1-E10, wherein at least 80 wt. % of the lipid composition (e.g., at least 85 wt. %, at least 90 wt. %, at least 95 wt. %) may comprise the octreotide or pharmaceutically acceptable salt thereof (and any other optional active ingredients) and lipid(s), phospholipid(s), solvents, and, if present, EDTA (allowing for any impurity inherent in these components). For example, at least 85 wt. % of the lipid composition (e.g., 86 wt. %, 87 wt. %, 88 wt. %, 89 wt. %) may comprise the octreotide (e.g., octreotide chloride), at least one lipid (e.g., glycerol dioleate), at least one phospholipid (e.g., phosphatidylcholine), at least one biocompatible organic solvent (e.g., ethanol and propylene glycol), and EDTA. These lipid compositions may have an initial (at release of the drug product) water of content of less than 2.0 wt. % (e.g., e.g., less than 1.0 wt. %, less than 0.9 wt. %, less than 0.8 wt. %, less than 0.7 wt. %, less than 0.6 wt. %, less than 0.5 wt. %).
E12. The method of any one of E1-E11, wherein at least 85 wt. % of the lipid composition (e.g., 86 wt. %, 87 wt. %, 88 wt. %, 89 wt. %) consists of octreotide chloride, glycerol dioleate, phosphatidylcholine, ethanol, and propylene glycol.
E13. The method of any one of E1-E12, wherein at least 86 wt. % (e.g., 87 wt. %, 88 wt. %, 89 wt. %) of the lipid composition consists of octreotide chloride, glycerol dioleate, phosphatidylcholine, ethanol, propylene glycol, EDTA, and the lipid composition has a water content of less than 1.0 wt. % (e.g., less than 0.9 wt. %, less than 0.8 wt. %, less than 0.7 wt. %, less than 0.6 wt. %, less than 0.5 wt. %) at the release of the lipid composition product for sale.
E13_1. The method of any one of E1-E13, wherein the lipid composition contains about 20 mg octreotide base/mL, about 6.5% w/w anhydrous ethanol, about 6.5% w/w propylene glycol, about 0.01% w/w ethylenediaminetetraacetic acid, about 0.01% w/w ethanolamine and soybean phosphatidylcholine/glycerol dioleate in a weight ratio 50/50 to final volume.
E13_2. The method of E13_1, wherein the volume of each administration is about 1 mL.
E14. The method of any one of E1-E13_2, wherein the lipid composition is administered by a syringe, a pre-filled syringe, an autoinjector, or a pen injector.
E15. The method of any one of E1-E14, wherein lipid composition is administered by subcutaneous injection.
E16. The method of any one of E1-E15, wherein the lipid composition is administered not more than once every two weeks, and in a volume of about 1 mL.
E17. The method of any one of E1-E16, wherein the patient has a maximum blood plasma concentration (Cₘₐₓ) of octreotide (at steady state) of less than 45 ng/ml, such as less than 40 ng/ml, less than 35 ng/ml, or less than 30 ng/ml, for each administration.
E18. The method of any one of E1-E17, wherein the patient has a maximum blood plasma concentration (Cₘₐₓ) of octreotide (at steady state) of 3-45 ng/ml, such as 4-40 ng/ml, such as 5-35 ng/ml, for each administration.
E19. The method of any one of E1-E18, wherein the patient has an average blood plasma concentration (CAV) of octreotide (at steady state) of 3-24 ng/ml, such as 4-20 ng/ml, such as 5-15 ng/ml or 6-10 ng/ml, for each administration.
E20. The method of any one of E1-E19, wherein the patient has an average blood plasma concentration (CAV) of octreotide (at steady state) of 4-20 ng/ml for each administration.
E21. The method of any one of E1-E20, the patient has a plasma concentration AUC of octreotide (at steady state) of 1300-6700 ng*h/ml, such as 1700-5000 ng*h/ml or 1700-3400 ng*h/ml, for each administration.
E22. The method of any one of E1-E21, wherein the patient has a plasma concentration AUC of octreotide (at steady state) of 1550-3500 ng*h/ml, such as 1600-3450 ng*h/ml, such as 1600-3400 ng*h/ml, for each administration.
E23. The method of any one of E1-E22, wherein administering the lipid composition comprising 10 or 20 mg of octreotide provides, in the patient, a drug depot comprising octreotide, said depot providing a release of octreotide to the patient over about two weeks and wherein the patient has blood plasma levels of octreotide of at least about 3 ng/ml, such as at least about 4 ng/ml, about 5 ng/ml, or about 6 ng/ml, for each administration.
E24. The method of any one of E1-E23, wherein the depot is in the subcutaneous tissue of the patient.
E25. The method of any one of E1-E24, wherein the lipid composition is administered to the patient once every 4 weeks+/−1 week (once every 28+/−7 days), e.g. for at least 24 weeks.
E26. The method of any one of E1-E25, wherein the patient has a maximum blood plasma concentration (Cₘₐₓ) of octreotide (at steady state) of less than 45 ng/ml, such as less than 40 ng/ml, less than 35 ng/ml, or less than 30 ng/ml, after each administration of the lipid composition.
E27. The method of any one of E1-E26, wherein the patient has a maximum blood plasma concentration (Cₘₐₓ) of octreotide (at steady state) of 3-45 ng/ml, such as 4-40 ng/ml, such as 5-35 ng/ml, after each administration of the lipid composition.
E28. The method of any one of E1-E27, wherein the patient has an average blood plasma concentration (CAV) of octreotide (at steady state) of 6-40 ng/ml, such as 7-30 ng/ml or 7-25 ng/ml, after each administration.
E29. The method of any one of E1-E28, wherein the patient has an average blood plasma concentration (CAV) of octreotide (at steady state) of 4-20 ng/ml after each administration.
E30. The method of any one of E1-E29, the patient has a plasma concentration AUC of octreotide (at steady state) of 1000-6700 ng*h/ml, such as 1000-5900 ng*h/ml, such a 1200-5000 ng*h/ml, or 1200-4200 ng*h/ml, after each administration.
E31. The method of any one of E1-E30, wherein the lipid composition is provided in a pre-filled syringe.
E32. The method of any one of E1-E31, wherein the lipid composition is provided in an autoinjector comprising a glass compartment containing the aforementioned lipid composition and/or any of its disclosed variations.
E33. The method of E32, wherein the glass compartment of the autoinjector is part of a pre-filled syringe.
E34. The method of any one of E1-E33, wherein the octreotide or a pharmaceutically acceptable salt thereof is provided in a kit for the administration, wherein the octreotide or a pharmaceutically acceptable salt is provided in one or more containers of the kit and is administered according to the aforementioned methods and/or any of its disclosed variations.
E35. The method of any one of E1-E34, wherein the administration of a lipid composition comprising 10 or 20 mg octreotide or a pharmaceutically acceptable salt thereof to a patient with acromegaly once every 28+/−7 days (once every 4 weeks+/−1 week), wherein the patient has IGF-1≤ULN (Upper limit of normal), as illustrated in FIG. 2; and optionally a mean GH cycle <2.5 ug/L, e.g., at week 24, and optionally with a p-value of about 0.0018.
E36. The method of any one of E1-E35, wherein the administration of a lipid composition comprising 10 or 20 mg octreotide or a pharmaceutically acceptable salt thereof to a patient with acromegaly once every 28+/−7 days (once every 4 weeks+/−1 week), wherein the patient experiences a biochemical response for at least 20 weeks, e.g., at least 25 weeks, such as illustrated in FIGS. 5 and 6.
E37. The method of any one of E1-E36, wherein the administration of a lipid composition comprising 10 or 20 mg octreotide or a pharmaceutically acceptable salt thereof to a patient with acromegaly once every 28+/−7 days (once every 4 weeks+/−1 week) provides an improved quality of life (QoL) compared to placebo, in total score, and e.g., in the physical domain score and/or the psychological domain score, e.g., as illustrated in FIG. 7.
E38. The method of any one of E1-E37, wherein the administration of a lipid composition comprising 10 or 20 mg octreotide or a pharmaceutically acceptable salt thereof to a patient with acromegaly once every 28+/−7 days (once every 4 weeks+/−1 week) provides an improved score on the treatment satisfaction questionnaire for medication n (TSQM), e.g., as illustrated in FIG. 8.
E39. The method of any one of E1-E38, wherein the administration of a lipid composition comprising 10 or 20 mg octreotide or a pharmaceutically acceptable salt thereof to a patient with acromegaly once every 28+/−7 days (once every 4 weeks+/−1 week) provides increased IGF-1 response rate, with about 8-10% differences, such as about 8.7% in the full patient population. The full population response rate went from about 49.7 to about 58.4%.
E40. The method of any one of E1-E39, wherein the administration of a lipid composition comprising 10 or 20 mg octreotide or a pharmaceutically acceptable salt thereof to a patient with acromegaly once every 28+/−7 days (once every 4 weeks+/−1 week) provides an increased IGF-1 response rate, with about 17-19% differences, such as about 18.3% in a new patient population. The new population response rate went from about 12.0 to about 30.3%. The naive population response rate went from about 20.2 to about 93.8%.
E41. The method of any one of E1-E40, wherein the administration of a lipid composition comprising 10 or 20 mg octreotide or a pharmaceutically acceptable salt thereof to a patient with acromegaly once every 28+/−7 days (once every 4 weeks+/−1 week) provides an improved IGF-1 response rate over 52 weeks.
E42. The method of any one of E1-E41, wherein the administration of a lipid composition comprising 10 or 20 mg octreotide or a pharmaceutically acceptable salt thereof to a patient with acromegaly once every 28+/−7 days (once every 4 weeks+/−1 week) provides reduced acromegaly symptom burden as measured by proportion of patients with any acromegaly symptom and the Acromegaly Index of Severity (AIS) score (sum of scores of the six acromegaly symptoms of headache, sweating, fatigue, joint pain, paresthesia, and soft tissue swelling).
E43. The method of any one of E1-E42, wherein the administration of a lipid composition comprising 10 or 20 mg octreotide or a pharmaceutically acceptable salt thereof to a patient with acromegaly once every 28+/−7 days (once every 4 weeks+/−1 week) provides increased patient and treatment satisfaction as measured by the Patient Satisfaction score and Treatment Satisfaction Questionnaire for Medication (TSQM) scores.
E44. The method of any one of E1-E43, wherein the administration of a lipid composition comprising 10 or 20 mg octreotide or a pharmaceutically acceptable salt thereof to a patient with acromegaly once every 28+/−7 days (once every 4 weeks+/−1 week) provides improved quality of life as measured by the Acromegaly Quality of Life Questionnaire (AcroQOL) scores and the EuroQOL 5D-5L VA.
E45. The method of any one of E1-E44, wherein the administration of a lipid composition comprising 10 or 20 mg octreotide or a pharmaceutically acceptable salt thereof to a patient with acromegaly once every 28+/−7 days provides a favorable safety profile and/or biochemical control, e.g., over an extended time period, e.g., over 52 weeks.

### Further Exemplary Embodiments

EA1. A lipid composition for treating acromegaly, comprising, consisting essentially of, or consisting of administering to a patient in need thereof a lipid composition comprising, consisting essentially of, or consisting of 10 or 20 mg of octreotide or a pharmaceutically acceptable salt thereof, wherein the lipid composition is administered to the patient once every 28+/−7 days (once every 4 weeks+/−1 week).
EA2. The composition of EA1, wherein the lipid composition is administered as a unit dose.
EA3. The composition of EA1 or EA2, wherein the lipid composition comprises 20 mg octreotide, or an equivalent amount of a pharmaceutically acceptable salt thereof.
EA4. The composition of any one of EA1-EA3, wherein the pharmaceutically acceptable salt thereof is hydrochloride (chloride).
EA5. The composition of EA4, wherein the octreotide, or pharmaceutically acceptable salt thereof is the sole active ingredient in the lipid composition.
EA6. The composition of any one of EA1-EA5, wherein the lipid composition further comprises, consists essentially of, or consists of glycerol dioleate, phosphatidylcholine, and ethanol.
EA7. The composition of any one of EA1-EA6, wherein the lipid composition comprises, consists essentially of, or consists of the octreotide or pharmaceutically acceptable salt thereof (e.g., octreotide chloride), glycerol dioleate, phosphatidylcholine, ethanol, and propylene glycol.
EA8. The composition of any one of EA1-EA7, wherein the lipid composition comprises, consists essentially of, or consists of the octreotide or pharmaceutically acceptable salt thereof (e.g., octreotide chloride), glycerol dioleate, phosphatidylcholine, ethanol, and EDTA.
EA9. The composition of any one of EA1-EA8, wherein the lipid composition comprises, consists essentially of, or consists of the octreotide or pharmaceutically acceptable salt thereof (e.g., octreotide chloride), glycerol dioleate, phosphatidylcholine, ethanol, propylene glycol, and EDTA.
EA10. The composition of any one of EA1-EA9, wherein the lipid composition comprises, consists essentially of, or consists of the octreotide or pharmaceutically acceptable salt thereof (e.g., octreotide chloride), glycerol dioleate, phosphatidylcholine, ethanol, propylene glycol, EDTA, ethanolamine and/or diethanolamine.
EA11. The composition of any one of EA1-EA10, wherein at least 80 wt. % of the lipid composition (e.g., at least 85 wt. %, at least 90 wt. %, at least 95 wt. %) may comprise the octreotide or pharmaceutically acceptable salt thereof (and any other optional active ingredients) and lipid(s), phospholipid(s), solvents, and, if present, EDTA (allowing for any impurity inherent in these components). For example, at least 85 wt. % of the lipid composition (e.g., 86 wt. %, 87 wt. %, 88 wt. %, 89 wt. %) may comprise the octreotide (e.g., octreotide chloride), at least one lipid (e.g., glycerol dioleate), at least one phospholipid (e.g., phosphatidylcholine), at least one biocompatible organic solvent (e.g., ethanol and propylene glycol), and EDTA. These lipid compositions may have an initial (at release of the drug product) water of content of less than 2.0 wt. % (e.g., e.g., less than 1.0 wt. %, less than 0.9 wt. %, less than 0.8 wt. %, less than 0.7 wt. %, less than 0.6 wt. %, less than 0.5 wt. %).
EA12. The composition of any one of EA1-EA11, wherein at least 85 wt. % of the lipid composition (e.g., 86 wt. %, 87 wt. %, 88 wt. %, 89 wt. %) consists of octreotide chloride, glycerol dioleate, phosphatidylcholine, ethanol, and propylene glycol.
EA13. The composition of any one of EA1-EA12, wherein at least 86 wt. % (e.g., 87 wt. %, 88 wt. %, 89 wt. %) of the lipid composition consists of octreotide chloride, glycerol dioleate, phosphatidylcholine, ethanol, propylene glycol, EDTA, and the lipid composition has a water content of less than 1.0 wt. % (e.g., less than 0.9 wt. %, less than 0.8 wt. %, less than 0.7 wt. %, less than 0.6 wt. %, less than 0.5 wt. %) at the release of the lipid composition product for sale.
EA13_1. The composition of any one of EA1-EA13, wherein the lipid composition contains about 20 mg octreotide base/mL, about 6.5% w/w anhydrous ethanol, about 6.5% w/w propylene glycol, about 0.01% w/w ethylenediaminetetraacetic acid, about 0.01% w/w ethanolamine and soybean phosphatidylcholine/glycerol dioleate in a weight ratio 50/50 to final volume.
EA13_2. The composition of EA13_1, wherein the volume of each administration is about 1 mL.
EA14. The composition of any one of EA1-EA13_2, wherein the lipid composition is administered by a syringe, a pre-filled syringe, an autoinjector, or a pen injector.
EA15. The composition of any one of EA1-EA14, the wherein lipid composition is administered by subcutaneous injection.
EA16. The composition of any one of EA1-EA15, wherein the lipid composition is administered not more than once every two weeks, and in a volume of about 1 mL.
EA17. The composition of any one of EA1-EA16, providing a maximum blood plasma concentration (Cₘₐₓ) of octreotide (at steady state) of less than 45 ng/ml, such as less than 40 ng/ml, less than 35 ng/ml, or less than 30 ng/ml, for each administration.
EA18. The composition of any one of EA1-EA17, providing a maximum blood plasma concentration (Cₘₐₓ) of octreotide (at steady state) of 3-45 ng/ml, such as 4-40 ng/ml, such as 5-35 ng/ml, for each administration.
EA19. The composition of any one of EA1-EA18, providing has an average blood plasma concentration (CAV) of octreotide (at steady state) of 3-24 ng/ml, such as 4-20 ng/ml, such as 5-15 ng/ml or 6-10 ng/ml, for each administration.
EA20. The composition of any one of EA1-EA19, providing an average blood plasma concentration (CAV) of octreotide (at steady state) of 4-20 ng/ml for each administration.
EA21. The composition of any one of EA1-EA20, providing a plasma concentration AUC of octreotide (at steady state) of 1300-6700 ng*h/ml, such as 1700-5000 ng*h/ml or 1700-3400 ng*h/ml, for each administration.
EA22. The composition of any one of EA1-EA21, providing a plasma concentration AUC of octreotide (at steady state) of 1550-3500 ng*h/ml, such as 1600-3450 ng*h/ml, such as 1600-3400 ng*h/ml, for each administration.
EA23. The composition of any one of EA1-EA22, wherein the administration provides a drug depot comprising octreotide in the patient, said depot providing a release of octreotide to the patient over about two weeks and wherein the patient has blood plasma levels of octreotide of at least about 3 ng/ml, such as at least about 4 ng/ml, about 5 ng/ml, or about 6 ng/ml, for each administration.
EA24. The composition of any one of EA23, wherein the depot is in the subcutaneous tissue of the patient.
EA25. The composition of any one of EA1-EA24, wherein the lipid composition is administered to the patient once every 4 weeks+/−1 week (once every 28+/−7 days), e.g. for at least 24 weeks.
EA26. The composition of any one of EA1-EA25, provides a maximum blood plasma concentration (Cₘₐₓ) of octreotide (at steady state) of less than 45 ng/ml, such as less than 40 ng/ml, less than 35 ng/ml, or less than 30 ng/ml, after each administration of the lipid composition.
EA27. The composition of any one of EA1-EA26, provides a maximum blood plasma concentration (Cₘₐₓ) of octreotide (at steady state) of 3-45 ng/ml, such as 4-40 ng/ml, such as 5-35 ng/ml, after each administration of the lipid composition.
EA28. The composition of any one of EA1-EA27, provides an average blood plasma concentration (CAV) of octreotide (at steady state) of 6-40 ng/ml, such as 7-30 ng/ml or 7-25 ng/ml, after each administration.
EA29. The composition of any one of EA1-EA28, provides an average blood plasma concentration (CAT) of octreotide (at steady state) of 4-20 ng/ml after each administration.
EA30. The composition of any one of EA1-EA29, provides a plasma concentration AUC of octreotide (at steady state) of 1000-6700 ng*h/ml, such as 1000-5900 ng*h/ml, such a 1200-5000 ng*h/ml, or 1200-4200 ng*h/ml, after each administration.
EA31. The composition of any one of EA1-EA30, provided in a pre-filled syringe.
EA32. The composition of any one of EA1-EA31, provided an autoinjector comprising a glass compartment containing the aforementioned lipid composition and/or any of its disclosed variations.
EA33. The composition of EA32, wherein the glass compartment of the autoinjector is part of a pre-filled syringe.
EA34. The composition of any one of EA1-EA33, provided in a kit, for the administration of octreotide or a pharmaceutically acceptable salt thereof, wherein the kit comprises one or more containers comprising 10 or 20 mg of octreotide or a pharmaceutically acceptable salt thereof, wherein the octreotide or a pharmaceutically acceptable salt in the one or more containers is administered according to the aforementioned method and/or any of its disclosed variations.
EA35. The composition of any one of EA1-EA34, wherein the composition is administrated to a patient with acromegaly once every 28+/−7 days (once every 4 weeks+/−1 week), wherein the patient has IGF-1≤ULN (Upper limit of normal), as illustrated in FIG. 2; and optionally a mean GH cycle<2.5 ug/L, e.g., at week 24, and optionally with a p-value of about 0.0018.
EA36. The composition of any one of E1-E35, disclosed herein is the administration of a lipid composition comprising 10 or 20 mg octreotide or a pharmaceutically acceptable salt thereof to a patient with acromegaly once every 28+/−7 days (once every 4 weeks+/−1 week), wherein the patient experiences a biochemical response for at least 20 weeks, e.g., at least 25 weeks, such as illustrated in FIGS. 5 and 6.
EA37. The composition of any one of E1-E36, disclosed herein, the administration of a lipid composition comprising 10 or 20 mg octreotide or a pharmaceutically acceptable salt thereof to a patient with acromegaly once every 28+/−7 days (once every 4 weeks+/−1 week) provides an improved quality of life (QoL) compared to placebo, in total score, and e.g., in the physical domain score and/or the psychological domain score, e.g., as illustrated in FIG. 7.
EA38. The composition of any one of EA1-EA37, disclosed herein, wherein the administration of the lipid composition to a patient with acromegaly once every 28+/−7 days (once every 4 weeks+/−1 week) provides an improved score on the treatment satisfaction questionnaire for medication n (TSQM), e.g., as illustrated in FIG. 8.
EA39. The composition of any one of EA1-EA38, wherein the administration of the lipid composition comprising 10 or 20 mg octreotide or a pharmaceutically acceptable salt thereof to a patient with acromegaly once every 28+/−7 days (once every 4 weeks+/−1 week) provides increased IGF-1 response rate, with about 8-10% differences, such as about 8.7% in the full patient population. The full population response rate went from about 49.7 to about 58.4%.
EA40. The composition of any one of EA1-EA39, wherein the administration of the lipid composition comprising 10 or 20 mg octreotide or a pharmaceutically acceptable salt thereof to a patient with acromegaly once every 28+/−7 days (once every 4 weeks+/−1 week) provides an increased IGF-1 response rate, with about 17-19% differences, such as about 18.3% in a new patient population. The new population response rate went from about 12.0 to about 30.3%. The naive population response rate went from about 20.2 to about 93.8%.
EA41. The composition of any one of EA1-EA40, wherein the administration of the lipid composition comprising 10 or 20 mg octreotide or a pharmaceutically acceptable salt thereof to a patient with acromegaly once every 28+/−7 days (once every 4 weeks+/−1 week) provides an improved IGF-1 response rate over 52 weeks.
EA42. The composition of any one of EA1-EA41, wherein the administration of the lipid composition comprising 10 or 20 mg octreotide or a pharmaceutically acceptable salt thereof to a patient with acromegaly once every 28+/−7 days (once every 4 weeks+/−1 week) provides reduced acromegaly symptom burden as measured by proportion of patients with any acromegaly symptom and the Acromegaly Index of Severity (AIS) score (sum of scores of the six acromegaly symptoms of headache, sweating, fatigue, joint pain, paresthesia, and soft tissue swelling).
EA43. The composition of any one of EA1-EA42, wherein the administration of the lipid composition comprising 10 or 20 mg octreotide or a pharmaceutically acceptable salt thereof to a patient with acromegaly once every 28+/−7 days (once every 4 weeks+/−1 week) provides increased patient and treatment satisfaction as measured by the Patient Satisfaction score and Treatment Satisfaction Questionnaire for Medication (TSQM) scores.
EA44. The composition of any one of EA1-EA43, wherein the administration of the lipid composition comprising 10 or 20 mg octreotide or a pharmaceutically acceptable salt thereof to a patient with acromegaly once every 28+/−7 days (once every 4 weeks+/−1 week) provides improved quality of life as measured by the Acromegaly Quality of Life Questionnaire (AcroQoL) scores and the EuroQOL 5D-5L VA.
EA45. The composition of any one of EA1-EA44, wherein the administration of a lipid composition comprising 10 or 20 mg octreotide or a pharmaceutically acceptable salt thereof to a patient with acromegaly once every 28+/−7 days (once every 4 weeks+/−1 week) provides a favorable safety profile and/or biochemical control, e.g., over an extended time period, e.g., over 52 weeks.

### Example 1 Clinical Trial Protocol Used Herein (Acroinnova 1)

### Overall Trial Design and Plan

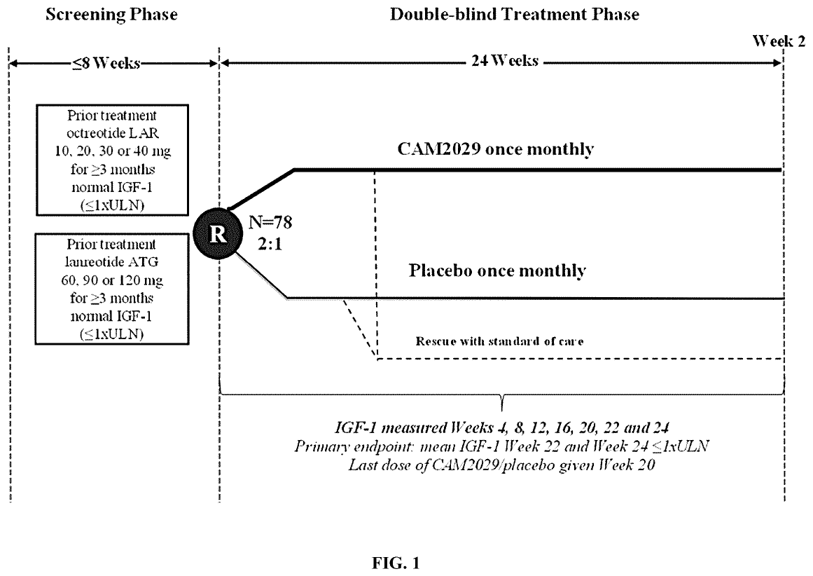

This is a Phase 3, randomized, double-blind, placebo-controlled, multi-center trial that assesses the efficacy and safety of CAM2029 versus placebo in patients with acromegaly. Eligible patients who are biochemically controlled on treatment with long-acting SSAs (octreotide or lanreotide) and who have prior evidence of active acromegaly disease will be randomized to treatment with either CAM2029 or placebo in a 24-week Double-blind Treatment Phase. An overview of the trial design is shown in FIG. 1.

### Screening Phase

The following patients will be eligible for inclusion in the trial (see further details in Section 5).

Patients with confirmed acromegaly on treatment with a stable dose of octreotide LAR (10 mg, 20 mg, 30 mg or 40 mg once monthly) for at least 3 months and with IGF-1 levels≤1 times LN and mean GH cycle levels<2.5 μg/L at screening.

Patients with confirmed acromegaly on treatment with a stable dose of lanreotide ATG (60 mg, 90 mg or 120 mg once monthly) for at least 3 months and with IGF-1 levels≤1×ULN and mean GH cycle levels<2.5 ug/L at screening.

The Screening Phase will have a duration of ≤8 weeks. During this period, the patients will visit the site at least 2 times to perform the screening procedures. The first dose of CAM2029 or placebo on Day 1 in the Double-blind Treatment Phase will be given 4 weeks (+3 days) after the last dose of octreotide LAR or lanreotide ATG.

### Double-Blind Treatment Phase

Patients who are eligible for the Double-blind Treatment Phase will be randomized in a 2:1 ratio to receive one of the following treatments:
CAM2029 once monthly for 24 weeks or
Placebo once monthly for 24 weeks

Randomization will be stratified based on previous treatment (octreotide LAR versus lanreotide ATG). Approximately 78 randomized patients are estimated to be needed for the double-blind phase in total, with 52 patients in the CAM2029 treatment arm and 26 patients in the placebo treatment arm.

The starting dose of CAM2029 is 20 mg once monthly, regardless of the patient's prior treatment and the previous octreotide LAR dose (10 mg, 20 mg, 30 mg or 40 mg) or lanreotide ATG dose (60 mg, 90 mg or 120 mg). Doses may be down-titrated to 10 mg CAM2029 (or corresponding placebo volume) based on safety and tolerability. After down-titration, the patient can return to the previous dose if the safety issue is resolved and the benefit-risk evaluation allows it. All dose adjustments must be discussed with the Medical Monitor prior to implementation.

During the 24-week Double-blind Treatment Phase, IGF-1 will be measured on Day 1 (before the first dose of CAM2029/placebo) and at Weeks 4, 8, 12, 16, 20, 22 and 24. The primary endpoint (IGF-1 levels≤1×ULN) will be evaluated as an average value of the measurements at Week 22 and Week 24. GH will be measured as random samples or cycles during the trial.

To maintain the integrity of the blind, patients, Investigators and the trial team will remain blinded to all individual IGF-1 and GH results until the trial is completed and a decision is made to unblind. IGF-1 results will be monitored by an independent reader during the trial. Patients should be discontinued from treatment with CAM2029/placebo and be switched to rescue with standard of care in case they experience worsening of signs and symptoms of acromegaly together with an increase in the levels of IGF-1 to ≥1.3×ULN at 2 consecutive visits. Patients who are switched to standard of care should continue participation in the trial and should follow all planned visits and assessments. The treatment allocation (CAM2029/placebo) of patients who are switched to standard of care will remain blinded until the trial is completed and a decision is made to unblind.

Safety will be evaluated continuously and plasma samples for assessment of octreotide concentration will be taken. Patient-reported treatment satisfaction will be assessed using a general questionnaire and will be compared between CAM2029 and placebo and with previous treatment. Health-related quality of life parameters will also be assessed.

Patients may self-administer CAM2029/placebo or have the IMP (investigational medicinal product) administered by their partner. The feasibility of self- or partner-administration of the IMP under the supervision of a trained trial personnel will be assessed. If the patients or their partners are considered competent by the trial personnel to administer the IMP, they may continue to administer the IMP monthly. If the IMP is not self- or partner-administered, the trial personnel will perform the administrations.

At Week 24, or 4 weeks after the last dose in case of premature withdrawal from the trial, the patients will come to the trial site for an End of Trial visit. Patients who complete the trial will be offered to continue with treatment with CAM2029 as roll-over patients in a long-term safety trial. Alternatively, patients will be switched back to their previous treatment for acromegaly.

### Rationale of Overall Trial Design and Choice of Control Group

### Trial Design

This Phase 3 trial is designed to assess the efficacy of CAM2029 versus placebo in patients with acromegaly. The primary efficacy objective will be assessed by the proportion of patients with biochemical control, defined as patients having a mean IGF-1 level≤1×ULN at Week 22 and Week 24 (assessed by a central laboratory; average of the 2 measurements).

Clinical guidelines for acromegaly management suggest evaluating the biochemical response to SSA treatment after at least 12 weeks of treatment. In addition, long-term response to SSAs has been demonstrated by normalization of GH/IGF-1 within the first 3 to 6 months of treatment. All patients who normalized their GH/IGF-1 levels after 3 months of treatment with IM octreotide LAR 20 mg once monthly (24 out of 56 enrolled patients) maintained biochemical control of the disease after 24 months of treatment.

The double-blind design minimizes the risk of potential bias. The double-blind period of 24 weeks in the current trial will allow comparison of efficacy and safety of CAM2029 versus placebo. IGF-1 levels are expected to be >1×ULN at Week 22 and Week 24 (i.e., 26 and 28 weeks after the last dose of octreotide LAR or lanreotide ATG) for patients on placebo treatment. The double-blind period of 24 weeks will ensure that the effect size captured at the Week 24 is attributable to CAM2029 and not to a confounder such as inactive disease due to the cumulative effect of past therapies (e.g., pituitary surgery, drugs or a combination of these). In a slowly progressive disease like acromegaly, which is usually diagnosed 5 to 10 years after the first symptom onset, a 24-week treatment with placebo can be considered a relatively short time frame with minimal or no negative impact on the overall disease history or patient disease outcome. Patients should be discontinued from treatment with the blinded IMP and be switched to rescue with standard of care in case they experience worsening of signs and symptoms of acromegaly together with an increase in the levels of IGF-1 to ≥1.3×ULN at 2 consecutive visits.

In line with recent recommendations, both patients with and without prior transsphenoidal surgery can be included in the trial, provided that at least 6 months have elapsed since the surgery.

### Selection of Endpoints

The choice of the primary endpoint (proportion of patients with mean IGF-1 levels≤1×ULN at Week 22 and Week 24) and secondary endpoints in this trial is consistent with the endpoints used in recent interventional trials (ClinicalTrials.gov identifier: NCT03252353; NCT02685709). According to clinical guidelines, IGF-1 is considered to be the most reliable biomarker of disease activity and for monitoring the efficacy of treatment in patients with acromegaly. In clinical practice, IGF-1 is the dominant driver for treatment decisions. IGF-1 is, hence, selected as the primary biomarker of efficacy in this Phase 3 trial. The primary objective of the current trial is to assess superiority of CAM2029 versus placebo in efficacy in terms of mean IGF-1 levels≤1×ULN at Week 22 and Week 24 (average of the 2 measurements).

The treatment effect on GH alone and combined with IGF-1 will also be assessed as secondary endpoints as both are considered biomarkers of disease control. GH circulating levels are subject to wide variation and GH levels are therefore usually calculated as mean levels (mean concentration of a 5-point profile within a 2-hour time period). In the current trial, both cycle and random values will be used for assessment of GH levels.

Because the CAM2029 product in a pre-filled syringe is planned to be provided for self- or partner-administration, the feasibility of self- or partner-administered injection of CAM2029 and convenience/patient treatment satisfaction will be evaluated as part of the objectives. Patient-reported treatment convenience with CAM2029 will also be assessed through a general questionnaire and through a patient satisfaction scale and will be compared to the previous treatment. Note: wherever normal range or ULN for IGF-1 is mentioned in the document (e.g., IGF-1≤1×ULN), it refers to values adjusted by age and sex.

### Treatments Administered

The treatments to be administered in this trial are summarized in 1.

TABLE 1
Trial treatments

| | | |
|---|---|---|
| Treatment | CAM2029 | Placebo |
| Treatment name | CAM2029 (octreotide | Placebo |
| | subcutaneous depot) | |
| Manufacturer | Fresenius Kabi Austria | Fresenius Kabi Austria |
| | GmbH | GmbH |
| Dosage | Solution for injection in a | Solution for injection in a |
| formulation | ready-to-use pre-filled | ready-to-use pre-filled |
| | syringe | syringe |
| Unit dose | 10 mg/0.5 mL for 10 mg | N/A |
| strengths | 20 mg/1.0 mL for 20 mg | |
| Dosage levels/ | 10 mg | 0.5 mL |
| volumes | 20 mg | 1.0 mL |
| Route of | SC injection | SC injection |
| administration | | |
| Packaging and | The IMP will be | The IMP will be |
| labelling | provided in pre-filled | provided in pre-filled |
| | syringes. Each pre-filled | syringes. Each pre-filled |
| | syringe will be labelled | syringe will be labelled |
| | in line with country | in line with country |
| | requirements | requirements |

IMP: investigational medicinal product;
N/A: not applicable;
SC: subcutaneous

The placebo product is identical to the CAM2029 product regarding appearance, volume and viscosity of the solutions.

All administrations of the IMP (CAM2029 or placebo) should be performed in the morning and as close as possible to the time point of the first dose. The dose, date and exact time of each IMP administration should be recorded. This applies for all visits, regardless of the pre-administration assessments (e.g., IGF-1, GH random, GH cycle, pre-administration questionnaires).

### Administration

CAM2029 will be administered as an SC injection in the abdomen and/or thigh, regardless of who is giving the injection (trial personnel, patient or partner). CAM2029 will be administered once monthly (every 28 days±7 days). If the date of a visit does not conform to the schedule, subsequent visits should be planned to maintain the visit schedule relative to the Day 1 visit. The injections should not be administered in a recently used injection site. The site of injection will be recorded. The dose, date and exact time of dosing must be recorded on the electronic case report form (eCRF).

All administrations of CAM2029 will be performed at the trial site. Self- or partner-administration of CAM2029 in the abdomen or thigh is allowed after appropriate training and under the supervision of trial personnel who has been adequately trained. The first self- or partner-administration should preferably be performed on Day 1. Trained trial personnel will document that the patient or partner understands the administration process, performs the injection correctly and administers a full dose. If the patients or their partners are considered competent to administer CAM2029, they may continue with the self- or partner-administration. If CAM2029 is not self- or partner-administered, the trial personnel will administer CAM2029.

### Doses and Dose Adjustments

If the patients are randomized to CAM2029, they will be treated with 20 mg CAM2029 on Day 1, regardless of their previous octreotide LAR dose (10 mg, 20 mg, 30 mg or 40 mg) or lanreotide ATG dose (60 mg, 90 mg or 120 mg). The first dose of 20 mg CAM2029 will be administered 4 weeks (±3 days) after the last dose of octreotide LAR or lanreotide ATG.

Doses of CAM2029 may be down-titrated from 20 mg to 10 mg once monthly for safety/tolerability reasons. After down-titration, the patient can return to the previous dose if the safety issue is resolved and the benefit-risk evaluation allows it. If the patient is on the lowest permitted dose (10 mg) and a further dose reduction for safety reasons is required, the patient will be discontinued from treatment and continue to be followed in the trial. All dose adjustments must be discussed with the Medical Monitor prior to implementation. For instructions regarding dose modifications and dose delays in case of adverse drug reactions or QTcF prolongations.

### Comparator Treatment (Placebo)

### Administration

Placebo will be administered as an SC injection in the abdomen and/or thigh, regardless of who is giving the injection (trial personnel, patient or partner). Placebo will be administered once monthly (every 28 days±7 days). If the date of a visit does not conform to the schedule, subsequent visits should be planned to maintain the visit schedule relative to the Day 1 visit. The injections should not be administered in a recently used injection site. The site of injection will be recorded. The dose, date and exact time of dosing must be recorded on the eCRF.

All administrations of placebo will be performed at the trial site. Self- or partner-administration of placebo in the abdomen or thigh is allowed after appropriate training and under the supervision of trial personnel who has been adequately trained. The first self- or partner-administration should preferably be performed on Day 1. Trained trial personnel will document that the patient or partner understands the administration process, performs the injection correctly and administers a full dose. If the patients or their partners are considered competent to administer placebo, they may continue with the self- or partner-administration. If placebo is not self- or partner-administered, the trial personnel will administer placebo.

### Doses and Dose Adjustments

If the patients are randomized to placebo, they will be treated with 1.0 mL placebo (corresponding to 20 mg CAM2029) on Day 1, regardless of their previous octreotide LAR dose (10 mg, 20 mg, 30 mg or 40 mg) or lanreotide ATG dose (60 mg, 90 mg or 120 mg). The first dose of 1.0 mL placebo will be administered 4 weeks (±3 days) after the last dose of octreotide LAR or lanreotide ATG.

The objectives of this trial and the corresponding endpoints are listed in table 2.

TABLE 2
Trial objectives and endpoints

| Objectives | Endpoints |
|---|---|
| Primary Objective | Primary Endpoint |
| To assess the superiority of CAM2029 | Proportion of patients with mean IGF-1 |
| compared to placebo in biochemical | levels ≤1xULN at Week 22 and Week 24 |
| response for IGF-1 | (average of the 2 measurements) |
| Secondary Objectives | Secondary Endpoints |
| To assess the superiority of CAM2029 | Proportion of patients with mean IGF-1 |
| compared to placebo in biochemical | levels ≤1xULN at Week 22 and |
| response for IGF-1 irrespective of dose of | Week 24, including patients who have |
| investigational medicinal product | their dose decreased to 10 mg CAM2029 |
| | (or 0.5 mL placebo) |
| To assess the superiority of CAM2029 | Proportion of patients with mean IGF-1 |
| compared to placebo in biochemical | levels ≤1xULN at Week 22 and Week 24 |
| response for IGF-1 and GH | and mean GH cycle levels <2.5 μg/L at |
| | Week 24 |
| To assess time to loss of response of | Time to IGF-1 levels >1xULN |
| CAM2029 compared to placebo | Time to IGF-1 levels ≥1.3xULN |
| To assess the efficacy of CAM2029 | Change from baseline to Week 24 in IGF- |
| versus placebo for IGF-1 levels over time | 1 levels |
| To assess the efficacy of CAM2029 | Change from baseline to Week 24 in GH |
| versus placebo for GH levels over time | levels |
| | Proportion of patients with mean GH |
| | cycle levels <2.5 μg/L at Week 24 |
| | Proportion of patients with mean GH |
| | cycle levels <1.0 μg/L at Week 24 |
| To assess use of rescue medication | Proportion of patients who receive rescue |
| | medication with standard of care |
| To evaluate the effects of CAM2029 and | Change in tumor size from screening to |
| placebo on tumor size | Week 24 |
| To assess the effects of CAM2029 and | Severity scores of clinical signs and |
| placebo on clinical signs and symptoms | symptoms of acromegaly over time |
| of acromegaly | Ring size over time |
| To measure patients' satisfaction with | TSQM scores over time using all |
| CAM2029 and placebo | 4 domains of TSQM (effectiveness, side |
| | effects, convenience and satisfaction) |
| | Patient satisfaction scale scores at Week |
| | 24 |
| To measure the effects of CAM2029 and | Change from baseline in AcroQoL and |
| placebo on quality of life | EQ-5D-5L scores |
| To measure patients' satisfaction with | Change from PRE module to the POST |
| self-administration of CAM2029 and | module in SIAQ scores |
| placebo | |
| To assess supervised self- or partner- | Proportion of patients/partners declared |
| administration of CAM2029 and placebo | competent by a healthcare professional to |
| | administer CAM2029 or placebo out of |
| | those trying |
| To assess the safety and tolerability of | Incidence of AEs and laboratory and ECG |
| CAM2029 and placebo | abnormalities |
| | Changes in laboratory values, vital signs, |
| | ECG readings and gallbladder imaging |
| To assess the local tolerability of | Incidence of injection site reactions |
| CAM2029 and placebo | (erythema and swelling) and level of |
| | injection site pain |
| To assess the plasma concentrations of | Octreotide plasma concentrations over |
| octreotide after administration of | time |
| CAM2029 in patients with acromegaly | |
| Exploratory Objectives | Exploratory Endpoints |
| To assess the treatment effects of | Prolactin levels |
| CAM2029 and placebo on prolactin | |
| To assess the immunogenicity of | Qualification and quantification of anti- |
| octreotide from CAM2029 in patients | octreotide antibodies |
| with acromegaly | |

AcroQoL: Acromegaly Quality of Life Questionnaire;
AE: adverse event;
ECG: electrocardiogram;
EQ-5D-5L: EuroQoL 5-dimension 5-level;
GH: growth hormone;
IGF-1: insulin-like growth factor-1;
SIAQ: Self-Injection Assessment Questionnaire;
TSQM: Treatment Satisfaction Questionnaire for Medication;
ULN: upper limit of normal

Dose volumes may be down-titrated from 1.0 mL to 0.5 mL once monthly (corresponding to 10 mg CAM2029) for safety/tolerability reasons. After down-titration, the patient can return to the previous dose volume if the safety issue is resolved and the benefit-risk evaluation allows it. If the patient is on the lowest permitted dose volume (0.5 mL) and a further dose reduction for safety reasons is required, the patient will be discontinued from treatment and continue to be followed in the trial. All dose adjustments must be discussed with the Medical Monitor prior to implementation. For instructions regarding dose modifications and dose delays in case of adverse drug reactions or QTcF prolongations

### Efficacy Assessments

### IGF-1

Serum levels of IGF-1 will be assessed at the time points. Pre-dose blood samplings for evaluation of IGF-1 efficacy endpoints will be done immediately before the IMP administration. In addition, post-dose samples will be taken at Week 20 for a separate evaluation of the PK/PD relationship. The date and exact time of IGF-1 sampling must be recorded in the eCRF. The IGF-1 samples will be analyzed by a central laboratory. Instructions for sampling and shipping of IGF-1 samples will be provided separately. Note: wherever normal range or ULN for IGF-1 is mentioned in the document (e.g., IGF-1≤1×ULN), it refers to age- and sex-adjusted values.

### GH

GH samples for evaluation of GH efficacy endpoints will be taken as pre-dose cycles (5 samples taken over a period of 2 hours, i.e., every 30 minutes), and as random pre-dose samples at the time points defined.

GH cycles will be assessed during 2 hours after 1 hour at rest in the morning before the IMP administration (120, 90, 60, 30 and 0 minutes before the administration). All GH cycles should be taken approximately at the same time as the first cycle.

GH random will be assessed after 1 hour at rest in the morning before the IMP administration.

The date and exact time of GH sampling must be recorded in the eCRF. The GH samples will be analyzed by a central laboratory. Sampling and shipping instructions will be provided separately.

Note: 0 minutes before administration should be understood as the planned time point for IGF-1 sampling, GH random sampling and other assessments to be performed immediately before IMP administration. Therefore, the 120-minute sampling should start 2 hours earlier (−120 minutes from time 0 minutes).

### Magnetic Resonance Imaging

Pituitary MRI scanning with gadolinium enhancement will be performed at screening and at Week 24/End of Trial. The scans will be assessed centrally to screen for pituitary tumor volume change.

If MRI scan with intravenous contrast (gadolinium) is contraindicated for a patient, a non-contrast MRI scan should be performed. If the MRI cannot be performed at all, a CT of the pituitary (with intravenous contrast if not contraindicated) may be performed. Regardless of imaging type, all regularly scheduled pituitary imaging will be centrally reviewed for consistency in the measurement of dimensions. The modality should remain consistent unless there is a development of a contraindication.

Historical MRI/CT scanning performed within 3 months before screening may be used as baseline measurement. If such an MRI/CT scanning is available, the screening MRI/CT does not need to be performed.

### Patient-Reported Outcomes

Patient-reported outcomes (PROs) for the assessment of convenience and treatment satisfaction include:
Treatment Satisfaction Questionnaire for Medication (TSQM) v1.4
Self-Injection Assessment Questionnaire (SIAQ) v2.0
Health-related quality of life questionnaires (Acromegaly Quality of Life Questionnaire [AcroQoL] and EuroQOL 5-dimension 5-level [EQ-5D-5L])

### Patient Satisfaction Scale

PROs will be completed in the patient's local language. The patient must be given enough space and time to complete the questionnaires.

The questionnaires should be completed per respective assessment schedule for each instrument and in the following order, as applicable: TSQM, SIAQ, AcroQOL, EQ-5D-5L and patient satisfaction scale.

The trial personnel will check the questionnaires for completeness and should ask the patient to complete any missing responses. Patient's refusal to complete all or any part of a questionnaire must be documented in the eCRF and should not be captured as a protocol deviation. The reason for non-completion of the questionnaire should be recorded in the eCRF. Original responses to the questionnaire are regarded as source data.

Completed questionnaires and any unsolicited comments written by the patient must be reviewed and assessed by the Investigator for responses that may indicate potential AEs or SAEs. This assessment must be documented in trial source records.

Investigators/trial personnel must not influence the completing of questionnaires by a patient through verbal or other non-verbal actions.

Treatment Satisfaction with Medication Questionnaire

The TSQM is a general PRO instrument designed to measure patient satisfaction with their medication. It has been previously used in patients with acromegaly to measure satisfaction with their medical treatment.

The TSQM Version 1.4 will be used to compare the patients' satisfaction with CAM2029 as compared to their previous treatment (octreotide LAR or lanreotide ATG) and assess satisfaction with CAM2029/placebo during the trial. The questionnaire has 4 domains (effectiveness, side effects, convenience and global satisfaction), each comprising 3 to 4 items. Responses are evaluated on a 5 to 7-point scale for each item. The calculation of domain scores will be done according to the instrument manual. All domain scores range from 0 to 100. Higher scores indicate a better outcome from the patient's perspective.

The TSQM questionnaire will be completed at the beginning of the scheduled trial visits, before the Investigator conducts any clinical assessment.

### Self-Injection Assessment Questionnaire

The SIAQ is an instrument designed to measure overall patient experience with SC self-administration. It comprises a PRE self-injection module containing 7 items and a POST self-injection module containing 21 items and will be completed by patients who choose to self-administer CAM2029 (or placebo).

The SIAQ PRE module will be completed by the patient before the first self-administration of CAM2029 (or placebo) and contains the following domains:
General feelings about injections
Self-confidence about giving self-injections
Satisfaction with the current way of taking medication

The SIAQ POST module will be completed 20 to 40 minutes after the first self-administration and after the self-administration at Week 20. The POST module contains the PRE domains plus the following domains:
Self-image
Injection site reactions
Ease of use of the self-injection device
Responses are evaluated on a 5- to 6-point scale for each item.

The calculation of domain scores will be done according to the instrument manual. All domain scores range from 0 to 10. Higher scores indicate a better patient experience.

### Health-Related Quality of Life

Health-related quality of life will be assessed by the AcroQOL questionnaire and utilities will be assessed using the EQ-5D-5L.

The AcroQOL is a disease-specific scale for assessment of quality of life in patients with acromegaly. The AcroQOL is uni-dimensional and contains 22 items divided into 2 scales; physical (8 items) and psychological (14 items). The psychological scale is further divided into 2 sub-scales: physical appearance and impact of the disease on personal relationships of the patient (7 items each). The calculation of domain scores will be done according to the instrument manual. Higher scores indicate better quality of life.

The EQ-5D-5L is a measure of health status from which utilities can be calculated for use in economic modeling. It has 2 components, the EQ-5D-5L descriptive system and the EQ-5D-5L visual analog scale (VAS). The EQ-5D-5L descriptive system comprises the following 5 dimensions: mobility, self-care, usual activities, pain/discomfort, and anxiety/depression. Each dimension has 5 levels: no problems, slight problems, moderate problems, severe problems and unable to do an activity or extreme problems. A utility index can be computed from the EQ-5D-5L descriptive system with utility scores ranging from −0.281 (worst imaginable health state) to 1 (best imaginable health state), with −0.281 representing an “unconscious” health state. The EQ-5D-5L VAS records the patient's self-rated health state on a 100-point vertical VAS (0=worst imaginable health state; 100=best imaginable health state).

### Patient Satisfaction Scale

At Week 24/End of Trial, the patients will complete a satisfaction questionnaire, in which they will rate their overall experience with CAM2029/placebo compared to their previous treatment with octreotide LAR or lanreotide ATG. The patients will evaluate the treatment on a 5-unit scale from “much worse” to “much better”.

### Clinical Signs and Symptoms of Acromegaly

The Investigator will measure the patient's ring size using the provided gauge at the time points. Ring size (circumference) will be measured at the fourth digit of the non-dominant hand. If a patient has a fourth digit size exceeding the highest size, the fifth digit of that hand will be used for initial and follow-up investigation.

At the same time points, the Investigator will also assess (together with the patient) the following symptoms of acromegaly on a 4-point score scale (none, mild, moderate and severe):
Headache
Sweating
Fatigue
Joint pain
Paresthesia
Soft tissue swelling

Worsening of signs and symptoms of acromegaly will be recorded as AEs at the discretion of the Investigator.

### Drug Concentration Assessments

### Blood Collection and Handling

Blood samples for evaluation of plasma concentrations of octreotide will be collected from all patients who receive at least one dose of IMP. Blood sampling will be performed at the time points.

All blood samples for analyses of octreotide will be taken by either direct venipuncture or indwelling cannula inserted in a forearm vein. Blood samples (2.5 mL) will be collected to yield 1 mL plasma for analysis of octreotide plasma concentration.

The date and exact time of dosing, as well as the date and exact time of blood sampling, must be recorded on the eCRF. Detailed instructions for the collection, handling and shipment of blood samples will be provided separately.

### Analytical Method

Plasma concentrations of octreotide will be measured using a validated liquid chromatography-tandem mass spectrometry assay with a lower limit of quantification (LLOQ) of approximately 0.0286 ng/mL.

### Results

The trial/example outlined above, i.e., 24-week, randomized, double-blind, placebo-controlled trial evaluating the efficacy and safety of CAM2029 in adults with acromegaly. CAM2029 designed for convenient, once-monthly (28+/−7 days) administration with ready-to-use syringe or injection pen to facilitate easy administration by patients. 72 patients were enrolled and on stable treatment with standard of care (SoC) with octreotide LAR or lanreotide ATG, who were randomized in a 2:1 ratio to treatment with octreotide SC depot or placebo. The trial met both the primary and the key secondary endpoints with statistical significance, including all sensitivity and supportive analyses.

Topline findings of the trial are summarized below for the intention-to-treat population:
Superior IGF-1 response rate (primary endpoint) of 72.2% in patients treated with octreotide SC depot versus 37.5% in patients treated with placebo (mean IGF-1≤upper limit of normal (ULN) week 22 and 24); p=0.0018
Superior IGF-1 and GH response rate (key secondary endpoint) of 70.0% in patients treated with octreotide SC depot versus 37.5% in patients treated with placebo (mean IGF-1≤ULN week 22 and week 24, and GH<2.5 μg/L week 24); p=0.0035
All sensitivity and supportive analyses confirmed the main findings. The response rate in the per protocol population was 81.0% vs. 38.1% for placebo; p=0.0002
Median time to loss of response (IGF-1>ULN) was not reached for patients treated with octreotide SC depot and was 8.4 weeks for placebo; p<0.0001
IGF-1 was well controlled until the end of the trial with a significant treatment difference versus placebo; p=0.0004
Increased quality of life (AcroQOL Total score) indicated from baseline to week 24 for octreotide SC depot vs. SoC; p=0.0038. Significant increases were seen for all AcroQOL domains for octreotide SC depot
Increased treatment satisfaction (incl. TSQM convenience score) from baseline to week 24 for octreotide SC depot vs. SoC; p<0.0001
Promising data obtained also for patient treatment satisfaction, e.g., improved patient satisfaction and treatment satisfaction convenience score for CAM2029, symptom control and quality of life (vs standard of care) were also achieved.

The trial included the following:
Patient demographics by treatment arm, table 3:

| | | | | | | OVERALL | |
|---|---|---|---|---|---|---|---|
| Parameter | Statistics or | CAM2029 | | PLACEBO | | (N = 72) | |
| (unit) | category | (N = 48) | | (N = 24) | | n (%) | |
| Age (years) | Mean (SD) | 57 | (11.2) | 52 | (15.1) | 55 | (12.8) |
| | Min-Max | 29-79 | | 20-82 | | 20-82 | |
| | 18-64, n (%) | 34 | (70.8) | 19 | (79.2) | 53 | (73.6) |
| | ≥65, n (%) | 14 | (29.2) | 5 | (20.8) | 19 | (26.4) |
| Sex (number) | Female n (%) | 28 | (58.3) | 12 | (50.0) | 40 | (55.6) |
| | Male n (%) | 20 | (41.7) | 12 | (50.0) | 32 | (44.4) |
| Weight (kg) | Mean (SD) | 85 | (17.6) | 87 | (17.3) | 86 | (17.4) |
| Height (cm) | Mean (SD) | 168 | (11.0) | 172 | (8.2) | 169 | (10.2) |
| BMI (kg/m²) | Mean (SD) | 30 | (5.6) | 30 | (5.8) | 30 | (5.6) |
| Region, n (%) | EU | 15 | (31.3) | 9 | (37.5) | 24 | (33.3) |
| | Europe, non-EU | 29 | (60.4) | 11 | (45.8) | 40 | (55.6) |
| | United States | 4 | (8.3) | 4 | (16.7) | 8 | (11.1) |

The trial enrolled adult patients with acromegaly on stable treatment with SoC, table 4

| | | CAM2029 | | PLACEBO | | OVERALL | |
|---|---|---|---|---|---|---|---|
| | | (N = 48) | | (N = 24) | | (N = 72) | |
| Parameter | Category | n (%) | | n (%) | | n (%) | |
| Time since | n | 47 | | 24 | | 71 | |
| Diagnosis (yrs) | | | | | | | |
| | Mean (SD) | 10.8 | (6.8) | 13.0 | (10.7) | 11.5 | (8.3) |
| Time since | <10 years, n (%) | 23 | (47.9) | 11 | (45.8) | 34 | (47.2) |
| Diagnosis | | | | | | | |
| | 10-20 years, n (%) | 19 | (39.6) | 9 | (37.5) | 28 | (38.9) |
| | ≥20 years, n (%) | 5 | (10.4) | 4 | (16.7) | 9 | (12.5) |
| Pituitary | Yes, n (%) | 42 | (87.5) | 21 | (87.5) | 63 | (87.5) |
| Surgery | No, n (%) | 6 | (12.5) | 3 | (12.5) | 9 | (12.5) |
| Treatment at | Octreotide LAR, | 25 | (52.1) | 14 | (58.3) | 39 | (54.2) |
| baseline | n (%) | | | | | | |
| | Lanreotide | 23 | (47.9) | 10 | (41.7) | 33 | (45.8) |
| | Autogel, n (%) | | | | | | |

### Example 2 Clinical Trial Protocol Used Herein (Acroinnova 2, HS-19-647), Open-Label, Single-Arm, Multi-Center Trial to Assess the Long-Term Safety of Octreotide Subcutaneous Depot (CAM2029) in Patients with Acromegaly

| | | |
|---|---|---|
| Treatment | CAM2029 pre-filled | CAM2029 pre-filled pen |
| | syringe | |
| Dosage | Solution for injection in a | Solution for injection in a |
| formulation | ready-to-use pre-filled | pre-filled pen |
| | syringe | |
| Unit dose | 10 mg/0.5 mL for 10 mg | 10 mg/0.5 mL for 10 mg |
| strengths | 20 mg/1.0 mL for 20 mg | 20 mg/1.0 mL for 20 mg |
| Dosage | 10 mg | 10 mg |
| levels | 20 mg | 20 mg |
| Route of | SC injection | SC injection |
| administration | | |
| Packaging and | CAM2029 will be provided | CAM2029 will be provided |
| labelling | in pre-filled syringes and | in pre-filled pens and will |
| | will be labelled in line | be labelled in line with |
| | with country requirements | country requirements |

This is a Phase 3, open-label, single-arm, multi-center trial that assesses the long-term safety of CAM2029 subcutaneous (SC) depot in patients with acromegaly, with one of the following

CAM2029 will be administered as an SC injection in the abdomen, thigh and/or buttock, regardless of who is giving the injection (trial personnel, patient or partner). CAM2029 will be administered once monthly (every 28 days±7 days). If the date of a visit does not conform to the schedule, subsequent visits should be planned to maintain the visit schedule relative to the Day 1 visit. The injections should not be administered in a recently used injection site. The site of injection will be recorded. The dose, date and exact time of dosing must be recorded on the electronic case report form (eCRF).

Self- or partner-administration of CAM2029 in the abdomen, thigh or buttock is allowed after appropriate training and under the supervision of adequately trained trial personnel. The first self- or partner-administration should preferably be performed on Day 1. Trained trial personnel will document that the patient or partner understands the administration process, performs the injection correctly and administers a full dose. Re-training will be performed for patients and/or partners switching from injections with pre-filled syringe to injections with pre-filled pen.

If the patients or their partners are considered competent to administer CAM2029, they may continue with the self- or partner-administration. If CAM2029 is not self- or partner-administered, the trial personnel will administer CAM2029. On Day 1 and at Weeks 4, 8, 12, 16, 20, 24, 36 and 48, the injections will be self- or partner-administered at the clinic. At Weeks 28, 32, 40 and 44, the patients may self-administer CAM2029 or receive the administration of CAM2029 by their partner at home. For these cases, the Investigator will dispense an appropriate number of investigational treatment packages for home administrations. The patients will record the dose(s), date(s) and exact time(s) of administration, the injection site and who performed the injection in a patient diary and will return the used devices at their next visit to the site. Patients will be asked to return all unused medication and packaging the latest at the completion of the trial or at the time of discontinuation of the investigational treatment to ensure proper drug accountability. Site staff will record in the appropriate documents the dates of the administration as well as confirmation of full dose administration. Detailed instructions will be provided separately.

### Extension Part of the Trial

CAM2029 will be administered every 28 days±7 days as an SC injection in the abdomen, thigh and/or buttock, regardless of who is giving the injection. The timing of each dose should be in relation to the date of the previous dose. Only the pre-filled pen will be used in the extension part of the trial. The injections should not be administered in a recently used injection site. The site of injection will be recorded. The dose, date and exact time of dosing must be recorded on the eCRF.

Training in how to administer CAM2029 with the pre-filled pen will be performed in connection with the first dose in the extension part of the trial (Day 1/Week 52) for patients/partners who used the pre-filled syringe in the main part of the trial. Patients or partners who used the pre-filled pen in the main part of the trial may also perform a re-training, if needed.

The Investigator will dispense an appropriate number of investigational treatment packages for administration of CAM2029. The patients will record the dose(s), date(s) and exact time(s) of administration, the injection site(s) and who performed the injection in a patient diary and will return the used devices at their next visit to the trial site. Patients will be asked to return all unused medication and packaging the latest at the completion of the trial or at the time of discontinuation of the investigational treatment to ensure proper drug accountability. Site staff will record in the appropriate documents the dates of the administration as well as confirmation of full dose administration. Detailed instructions will be provided separately.

It is important that patients contact the Investigator/site staff in case they are experiencing any AE/SAEs or have any concerns in connection with the administration of CAM2029.

Approximately 70 patients (new) will be enrolled directly into the main part of this trial from the beginning, i.e., starting with the screening period. These patients are responsive to SSA treatment but either: inadequately controlled on standard doses, i.e., they have mean IGF-1 levels at screening >1×ULN and ≤2.0×ULN with or without prior pituitary radiotherapy, or adequately controlled on standard doses, i.e., they have mean IGF-1 levels≤1×ULN at screening, either without prior pituitary radiotherapy or with a history of pituitary radiotherapy at least 3 years prior to screening and confirmed disease activity.

In addition, approximately 70 patients from the preceding trial (Example 1, Acroinnova 2), if agreed and consented, will continue to participate as roll-over patients in the main part of this trial, during which they will be treated with CAM2029. Patients rolling over from the preceding trial will only participate in the last 6 months of the trial, starting with Week 24 and ending at Week 52. Their last visit (Week 24) in trial the trial in Example 1 will be their first visit in this long-term safety trial. Therefore, the Week 24 visits in both trials will generally be the same.

Roll-over patients from the preceding trial (Example 1) will, at their last visit (Week 24/End of Trial [EOT]) in the preceding trial, be offered to continue participation in this trial. If these patients fulfill selected eligibility criteria and consent to continue their participation in this trial, their last visit in the preceding trial will be considered as the Week 24 visit of the trial. These roll-over patients will be treated with CAM2029 from Week 24 to Week 52

### New Patients

The following patients will be eligible for inclusion in the trial:

Patients with confirmed acromegaly on treatment with a stable dose of octreotide long-acting release (LAR; 10 mg, 20 mg, 30 mg or 40 mg once monthly) or lanreotide autogel (ATG: 60 mg, 90 mg or 120 mg once monthly) for at least 3 months and with IGF-1 levels>1×ULN and ≤2.0×ULN at screening with or without prior pituitary radiotherapy (at least 3 years prior to screening) or IGF-1 levels≤1×ULN at screening, in patients who either have not had prior pituitary radiotherapy or have a history of pituitary radiotherapy at least 3 years prior to screening and confirmed disease activity defined as IGF-1 levels>1×ULN during the period from 3 to 9 months prior to screening.

The Screening Phase will have a duration of ≤8 weeks. During this period, the patients will visit the site at least 2 times to perform the screening procedures. The first dose of CAM2029 on Day 1 in the Open-label Treatment Phase will be given 4 weeks (±3 days) after the last dose of octreotide LAR or lanreotide ATG.

### Open-Label Treatment Phase

Roll-Over Patients from Trial Acroinnova 1

Patients in trial Acroinnova 1 who consent to participate in the long-term safety trial will receive 20 mg or 10 mg CAM2029 at Week 24, depending on the CAM2029 dose (or corresponding volume of placebo) they were treated with at the end of the trial. Patients who were switched to standard of care during trial HS-18-633 due to worsening of the acromegaly will at Week 24 be offered to continue in trial Acroinnova 2 on 20 mg CAM2029.

### New Patients

The starting dose of CAM2029 is 20 mg once monthly, regardless of the patient's prior treatment and the previous octreotide LAR dose (10 mg, 20 mg, 30 mg or 40 mg) or lanreotide ATG dose (60 mg, 90 mg or 120 mg).

### All Patients

During treatment in the Open-label Treatment Phase, doses of CAM2029 may be down-titrated from 20 mg to 10 mg based on safety and tolerability. After down-titration, the patient can return to the previous dose if the safety issue is resolved and the benefit-risk evaluation allows it.

Safety variables will be evaluated throughout the trial. Patients who discontinue treatment will continue to be followed in the trial until Week 52.

During this trial, patients may self-administer CAM2029 or have CAM2029 administered by their partner, starting with the first dose on Day 1. The feasibility of self- or partner-administration of CAM2029 under the supervision of trained trial personnel will be assessed. If the patients or their partners are considered competent by the trial personnel to administer CAM2029, they may continue to administer CAM2029 monthly. If CAM2029 is not self- or partner-administered, the trial personnel will perform the administration.

Circulating octreotide, IGF-1 and GH levels will be assessed throughout this trial. Patient-reported treatment convenience and health-related QoL parameters will also be assessed.

At Week 52 (or 4 weeks after the last dose in case of premature withdrawal from the trial), the patients will come to the trial site for an EOT visit. All patients who complete the trial will be switched back to their previous treatment for acromegaly unless they proceed directly to the extension part of the trial.

### Extension Part of the Trial

The 52-week extension part of the trial is added to enable further collection of long-term safety and PRO data on CAM2029, and to provide an opportunity for patients who have completed treatment with CAM2029 in the main part of the trial to continue treatment with CAM2029. The extension part will include both patients who continue directly from the main part of the trial, and re-invited patients who have previously completed the HS-19-647 trial but who completed the main part more than 4 weeks ago or were treated with standard of care in between the 2 parts (regardless of the time elapsed since the main part of HS-19-647 was completed). For patients who continue directly from the main part of the trial, their last visit (Week 52) in the main part will be their first visit in the extension part of the trial. For the re-invited patients, there will be a screening period before treatment can be initiated in the extension part of the trial.

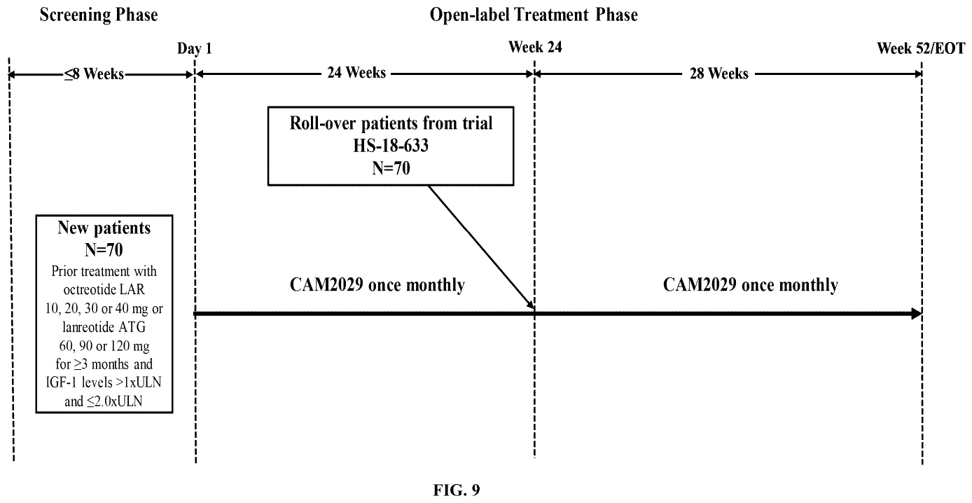

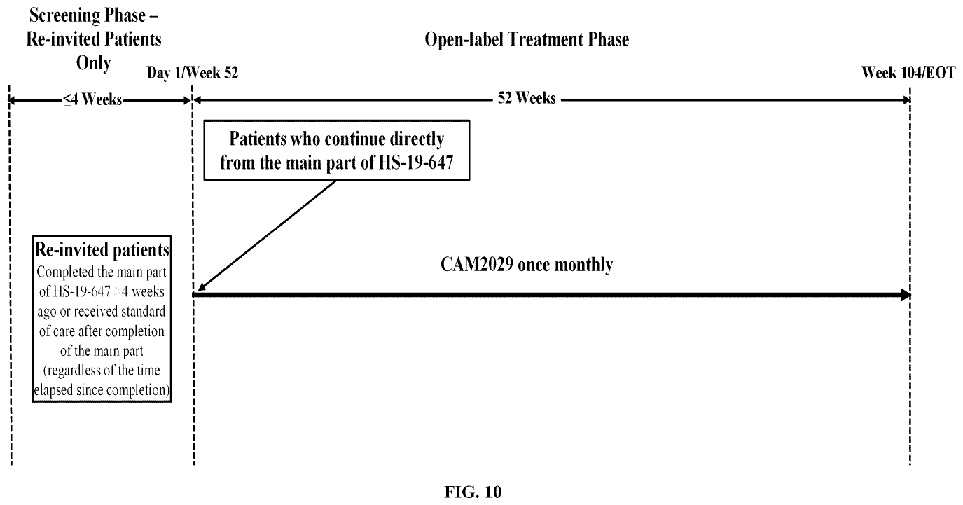

FIGS. 9 and 10 illustrates the overall clinical trial of Acroinnova 2.

Description of parts of the trial.

### Screening Phase

Patients who Continue Directly from the Main Part of the Trial

For patients who continue directly from the main part of the trial, the last visit in the main part of the trial (Week 52) will be considered the first visit in the extension part of the trial. Assessments performed at the Week 52 visit do not need to be repeated at the Day 1 visit in the extension part of the trial, provided that the assessments have been performed within the last 4 weeks.

Re-Invited Patients from Acroinnova 2

Patients who previously participated in the Acroinnova 2 trial but who completed treatment in the trial more than 4 weeks ago or who were switched back to standard of care (regardless of the time elapsed since completing the trial), will undergo a brief screening before treatment in the extension part of the trial can be initiated. There are no restrictions as to which standard of care they received (e.g., monotherapy or combination therapy), which dose levels or dosing frequencies of standard of care they were on, or how long they received standard of care in the period between the 2 parts of the trial.

### Open-Label Treatment Phase

Patients who participate in the extension part of the trial will receive treatment with 20 mg or 10 mg CAM2029 (depending on the dose they received at the end of the main part of the trial) using the pre-filled pen throughout the 52-week treatment period. In connection with the first dose on Day 1/Week 52, there will be a possibility for training in using the pre-filled pen for patients/partners who used the pre-filled syringe in the main part of the trial. Patients or partners who used the pre-filled pen in the main part of the trial may also perform a re-training, if needed.

Safety variables will be evaluated throughout the extension part of the trial. Circulating IGF-1 and GH levels will also be assessed, as well as patient reported treatment convenience, health economics and health-related QOL parameters.

At Week 104 (or 4 weeks after the last dose in case of premature withdrawal from the trial), the patients will come to the trial site for an EOT visit. All patients who complete the extension part of the trial will then continue to be treated for their acromegaly according to the Investigator's discretion.

### Trial Treatment

CAM2029 20 mg (1.0 mL, pre-filled syringes or pre-filled pens) will be administered through SC injections in the abdomen, thigh or buttock once monthly (28 days±7 days). The dose may be decreased to 10 mg (0.5 mL administered in pre-filled syringes or pre-filled pens) based on safety and tolerability. After down-titration, the patient can return to the previous dose if the safety issue is resolved and the benefit-risk evaluation allows it. From the first dose on Day 1 in the main part of the trial, CAM2029 may be self- or partner-administered. If CAM2029 is not self- or partner-administered, the trial personnel will administer CAM2029. The patients are adults, male of female, with acromegaly.

### Main Inclusion Criteria—Main Part of the Trial

For Roll-over Patients from Trial HS-18-633 (Acroinnova 1)

Patients who attend the Week 24 visit of the preceding trial (HS-18-633) and are willing to continue their participation in this trial

### For New Patients

Diagnosis of acromegaly by historical evidence of (persistent or recurrent) acromegaly as documented by:
IGF-1 levels>1×ULN,
Pituitary adenoma on magnetic resonance imaging (MRI) or pathology report. Computerized tomography is accepted if MRI could not be performed,
IGF-1 levels>1×ULN measured at least 3 months after the surgery for patients who have undergone pituitary surgery,
Treatment with a stable dose of octreotide LAR (10 mg, 20 mg, 30 mg or 40 mg) or lanreotide ATG (60 mg, 90 mg or 120 mg) for at least 3 months as monotherapy prior to screening,
IGF-1 levels>1×ULN and ≤2.0×ULN at screening (adjusted for age and sex; mean value of the first measurement at screening and the second measurement at 2 weeks before Day 1) with or without prior pituitary radiotherapy (at least 3 years prior to screening), or IGF-1 levels≤1×ULN at screening (adjusted for age and sex; value of the first measurement at screening and the second measurement at 2 weeks before Day 1), either without prior pituitary radiotherapy or with prior pituitary radiotherapy at least 3 years prior to screening and confirmed disease activity defined as IGF-1 levels>1×ULN during the period from 3 to 9 months prior to screening,
Adequate liver, pancreatic, renal and bone marrow functions,
Normal ECG.

### Main Exclusion Criteria—Main Part of the Trial

For Roll-over Patients from Trial HS-18-633

Unresolved, drug-related serious adverse event (SAE) from the preceding trial (HS-18-633)

Patients with a clinically significant or unstable medical or surgical condition that may preclude safe and complete trial participation.

### For New Patients

Have received medical treatment for acromegaly with pasireotide (within 6 months prior to screening), pegvisomant (within 3 months prior to screening), dopamine agonists (within 3 months prior to screening) or other investigational agents (within 30 days or 5 half-lives prior to screening [whichever is longer]).

Patients who usually take octreotide LAR or lanreotide ATG less frequently than every 4 weeks (e.g., every 6 weeks or 8 weeks).

Patients with compression of the optic chiasm causing any visual field defect for whom surgical intervention is indicated.

Patients who have undergone major surgery/surgical therapy for any cause within 1 month prior to screening.

Patients who have undergone pituitary surgery within 6 months prior to screening.

Patients who have received prior pituitary irradiation within 3 years prior to screening.

Patients with poorly controlled diabetes mellitus (hemoglobin A1c [HbA1c]>8.0%).

### Continuation Criteria for Patients Who Continue Directly to the Extension Part of the Trial

Patients who continue directly from the main part of the trial must complete treatment with CAM2029 in the main part of the trial, attend the Week 52 visit, and provide written informed consent to continue treatment in the extension part of the trial before treatment can be continued.

### Main Inclusion Criteria for Re-Invited Patients—Extension Part of the Trial

Completed treatment with CAM2029 in the main part of the trial and attended the Week 52 visit.

Adequate liver, pancreatic and renal functions.

### Normal ECG

### Main Exclusion Criteria for Re-Invited Patients—Extension Part of the Trial

Receiving treatments (other than treatments for acromegaly) known to affect GH or IGF-1 concentration.

Patients who have undergone major surgery/surgical therapy (including pituitary surgery) for any cause within 1 month prior to screening.

Patients who have received pituitary irradiation since the end of the main part of the trials.

Patients with poorly controlled diabetes mellitus (HbA1c>8.0%).

### Assessments of Safety and Efficacy Variables and Drug Concentration

### Assessments of Safety Variables

Safety will be evaluated throughout both parts of the trial by a recording of AEs, vital signs, ECGs and gallbladder ultrasounds and collection of safety laboratory samples. In the main part of the trial, local tolerability at the injection sites will also be evaluated and blood samples for assessment of prolactin levels and presence of anti-drug antibodies will be taken.

### Adverse Events

AEs will be assessed from the time of informed consent until completion of all trial procedures and discharge from the trial (both parts of the trial).

### Vital Signs

Vital signs consist of body temperature, blood pressure (systolic and diastolic), pulse rate and respiratory rate and will be collected throughout both parts of the trial following a resting period of at least 3 minutes.

### ECGs

Triplicate 12-lead ECG (3 ECG recordings at approximately 2-minute intervals) will be performed at scheduled time points during both parts of the trial. ECGs will be recorded after the patient has been resting in a supine position for at least 10 minutes. All ECGs should be recorded with the patient in the same physical position.

### Gallbladder Ultrasounds

A gallbladder ultrasound will be performed locally at the visits at screening, and at Weeks 24 and 52 in the main part of the trial and Weeks 76 and 104 in the extension part of the trial. Patients with symptomatic cholelithiasis within 3 months prior to screening for the main part of the trial will be excluded from participating in the trial. Information on the presence of gallstones or gallbladder sludge will be recorded on the appropriate electronic case report form page.

### Clinical Safety Laboratory

Clinical safety laboratory assessments (including biochemistry, hematology, coagulation, thyroid stimulating hormone, HbA1c, serological markers [human immunodeficiency virus and hepatitis B and C], vitamin B₁₂ and urinalysis) will be performed throughout both parts of the trial. A central laboratory will be used for the analysis of all laboratory evaluations.

### Local Tolerability at the Injection Sites

The Investigator will assess the degree of local tolerability in terms of erythema and swelling at the injection site throughout the main part of the trial. The patient will also self-assess the injection site pain using a numerical rating scale at the same time points. The degree of severity of all parameters will be collected. Assessments will be done immediately and 1 hour after the injections. Any event that occurs or persists beyond 1 hour after the injection will be collected as an AE.

### Assessments of Efficacy Variables

IGF-1 will be assessed throughout both parts of the trial. Pre-dose blood samplings for evaluation of IGF-1 efficacy endpoints in the main part of the trial will be done immediately before the CAM2029 administration. In addition, post-dose samples will be taken for a separate evaluation of the pharmacokinetic (PK)/pharmacodynamic (PD) relationship in the main part of the trial. The IGF-1 samples will be analyzed by a central laboratory.

In the main part of the trial, GH samples for evaluation of GH efficacy endpoints will be taken as pre-dose cycles (5 samples taken over a period of 2 hours, i.e., every 30 minutes) and as random pre-dose samples at different time points. In the extension part of the trial, GH samples will be taken as cycles and as random samples at approximately the same time points as the first sample in the extension part. The GH samples will be analyzed by a central laboratory.

Pituitary MRI (or computerized tomography if MRI is contraindicated or not available) will be performed at screening and at Weeks 52 and 104.

### Patient-Reported Outcomes

Patient-reported outcomes (PROs) for the assessment of convenience and treatment satisfaction include the TSQM v1.4 (both parts of the trial), the SIAQ v2.0 (main part of the trial), a patient satisfaction scale (main part of the trial), the health-related QOL questionnaires AcroQOL and EQ-5D-5L (both parts of the trial) and SF-36 (extension part of the trial), and the health economic outcomes questionnaire WPAI (extension part of the trial). The PROs will be completed at various time points during the trial.

Treatment Satisfaction with Medication Questionnaire (TSQM)

The TSQM is a general PRO instrument designed to measure patient satisfaction with their medication. It has been previously used in patients with acromegaly to measure satisfaction with their medical treatment. The TSQM Version 1.4 will be used to compare the patients' satisfaction with CAM2029 as compared to their previous treatment (octreotide LAR or lanreotide ATG) and assess satisfaction with CAM2029 during both parts of the trial. The questionnaire has 4 domains (effectiveness, side effects, convenience and global satisfaction), each comprising 3 to 4 items. Responses are evaluated on a 5- to 7-point scale for each item. The calculation of domain scores will be done according to the instrument manual. All domain scores range from 0 to 100. Higher scores indicate a better outcome from the patient's perspective. The TSQM questionnaire will be completed at the beginning of the scheduled trial visits before the Investigator conducts any clinical assessment.

### Self-Injection Assessment Questionnaire (SIAQ)

The SIAQ is an instrument designed to measure overall patient experience with SC self-administration. It comprises a PRE self-injection module containing 7 items and a POST self-injection module containing 21 items and will be completed by patients who choose to self-administer CAM2029 in the main part of the trial.

The SIAQ PRE module will be completed by the patient before the first self-administration of CAM2029 and contains the following domains:
General feelings about injections
Self-confidence about giving self-injections
Satisfaction with the current way of taking medication

The SIAQ POST module will be completed 20 to 40 minutes after the first self-administration and after the self-administration. The POST module contains the PRE domains plus the following domains:
Self-image
Injection site reactions
Ease of use of the self-injection device

Responses are evaluated on a 5- to 6-point scale for each item.

The calculation of domain scores will be done according to the instrument manual. All domain scores range from 0 to 10. Higher scores indicate a better patient experience.

### Health-Related Quality of Life

Health-related QOL will be assessed by the AcroQOL questionnaire (both parts of the trial) and the SF-36 questionnaire (extension part of the trial), and utilities will be assessed using the EQ-5D-5L (both parts of the trial)

The AcroQOL is a disease-specific scale for assessment of QoL in patients with acromegaly. The AcroQOL is uni-dimensional and contains 22 items divided into 2 scales; physical (8 items) and psychological (14 items). The psychological scale is further divided into 2 sub-scales: physical appearance and impact of the disease on personal relationships of the patient (7 items each). The calculation of domain scores will be done according to the instrument manual. Higher scores indicate better QoL.

The SF-36 is a widely used standardized instrument with strong psychometric properties. The SF-36 will be used in the extension part of the trial to assess self-perceptions of general health functioning across multiple dimensions (including general, physical, and emotional/psychiatric functioning). Higher scores indicate better quality of life. The SF-36 can be used in clinical practice and research, health policy evaluations, and general population surveys. It comprises a physical component summary and a mental component summary, both for which a higher score indicates better health status.

The EQ-5D-5L is a measure of health status from which utilities can be calculated for use in economic modeling. It has 2 components, the EQ-5D-5L descriptive system and the EQ-5D-5L visual analog scale (VAS). The EQ-5D-5L descriptive system comprises the following 5 dimensions: mobility, self-care, usual activities, pain/discomfort, and anxiety/depression. Each dimension has 5 levels: no problems, slight problems, moderate problems, severe problems and unable to do an activity or extreme problems. A utility index can be computed from the EQ-5D-5L descriptive system with utility scores ranging from −0.281 (worst imaginable health state) to 1 (best imaginable health state), with −0.281 representing an “unconscious” health state. The EQ-5D-5L VAS records the patient's self-rated health state on a 100-point vertical VAS (0=worst imaginable health state; 100=best imaginable health state). Scorings will be derived as previously described.

### Patient Satisfaction Scale

At Week 24 and Week 52/EOT in the main part of the trial, the patients will complete a satisfaction questionnaire, in which they will rate their overall experience with CAM2029 compared to their previous treatment with octreotide LAR or lanreotide ATG. The patients will evaluate the treatment on a 5-unit scale from “much worse” to “much better”.

### Work Productivity and Activity Impairment (WPAI)

WPAI will be assessed in the extension part of the trial. The WPAI is an instrument to measure impairments in both paid work and unpaid work. It measures absenteeism, presenteeism as well as impairments in unpaid activity because of health problem during the past 7 days. It has been validated to quantify work impairments for numerous diseases, such as asthma, psoriasis, irritable bowel syndrome, ankylosing spondylitis and Crohn's disease. In addition, the WPAI questionnaire has been used to compare work impairments between treatment groups in clinical trials or between subjects with different disease severity levels.

The general health WPAI consists of 6 questions:
1=Currently employed
2=Hours missed due to health problems
3=Hours missed other reasons
4=Hours actually worked
5=Degree health affected productivity while working (using a 0 to 10 VAS)
6=Degree health affected productivity in regular unpaid activities (VAS)

The recall period for questions 2 to 6 is 7 days. Four main outcomes can be generated from the WPAI and expressed in percentages by multiplying the following scores by 100, with higher percentages indicating greater work productivity loss and activity impairment:
1) Percent work time missed due to health=Q2/(Q2+Q4) for those who were currently employed
2) Percent impairment while working due to health=Q5/10 for those who were currently employed and actually worked in the past 7 days
3) Percent overall work impairment due to health Q2/(Q2+Q4)+((1−Q2/(Q2+Q4))×(Q5/10)) for those who were currently employed
4) Percent activity impairment due to health Q6/10 for all respondents

For those who missed work and did not actually work in the past 7 days, the percent overall work impairment due to health will be equal to the percent work time missed due to health.

### Clinical Signs and Symptoms of Acromegaly

The Investigator will measure the patient's ring size using the provided gauge at many time points. Ring size (circumference) will be measured at the fourth digit of the non-dominant hand. If a patient has a fourth digit size exceeding the highest size, the fifth digit of that hand will be used for initial and follow-up investigation.

At the same time points, the Investigator will also assess (together with the patient) the following symptoms of acromegaly on a 4-point score scale (none, mild, moderate and severe):
Headache
Sweating
Fatigue
Joint pain
Paresthesia
Soft tissue swelling

Worsening of signs and symptoms of acromegaly will be recorded as AEs at the discretion of the Investigator.

Ring size and severity of symptoms of acromegaly (headache, sweating, fatigue, joint pain, paresthesia and soft tissue swelling) will be assessed throughout both parts of the trial.

### Drug Concentration Assessments

Blood samples for assessment of plasma concentrations of octreotide will be taken during the main part of the trial. All blood samples for analyses of octreotide will be taken by either direct venipuncture or indwelling cannula inserted in a forearm vein. Blood samples (2.5 mL) will be collected to yield 1 mL plasma for analysis of octreotide plasma concentration.

Plasma concentrations of octreotide will be measured using a validated liquid chromatography-tandem mass spectrometry assay with a lower limit of quantification (LLOQ) of approximately 0.0286 ng/mL

### Other Assessments

Patients and their partners will have the possibility to administer CAM2029 by themselves (after appropriate training). Qualified trial personnel will supervise the administration and assess the patient's/partner's ability to successfully administer CAM2029.

### Statistical Methods (Data Analysis)

### General Considerations

The trial consists of 3 different patient groups; 2 patient groups previously treated with either CAM2029 (Group A) or placebo (Group B) in the double-blind trial (HS-18-633) and one group of patients who are new to treatment with CAM2029 in the current trial (Group C). In general, descriptive statistics will be presented for the 3 groups separately, Groups A and C together, and overall. Data will also be presented by trial part (main and extension) and overall, where applicable.

Safety analyses will be based on the safety analysis set, the full exposure safety analysis set and the continuous full exposure safety analysis set.

All efficacy analyses will be based on the intention-to-treat (ITT) analysis set. For all efficacy endpoints, descriptive measures and 95% confidence interval (CI) for the difference between Group A and Group B will be presented, unless otherwise specified. Group C will also be presented descriptively.

For roll-over patients who were randomized in the preceding trial HS-18-633, data collected in that trial will be used in the analyses and summaries of the current trial as appropriate.

### Analysis Sets

ITT Analysis Set: All patients who have been treated with CAM2029 or placebo in Groups A and B (in the case of roll-over patients from HS-18-633) or CAM2029 in Group C (in the case of new patients).

Per Protocol (PP) Analysis Set: All patients in the ITT with no major protocol deviations that would have an impact on the efficacy assessment.

Safety Analysis Set: All patients who were administered at least one dose of CAM2029 or placebo in Groups A and B (in the case of roll-over patients from HS-18-633) or at least one dose of CAM2029 in Group C (in the case of new patients).

Full Exposure Safety Analysis Set: All patients who have been exposed to CAM2029 for at least 52 weeks.

Continuous Full Exposure Safety Analysis Set: All patients who have been continuously exposed to CAM2029 for at least 52 weeks.

### Safety Analyses

An overview of all treatment-emergent AEs (TEAEs) including severity, relationship to the investigational medicinal product, SAEs and TEAEs leading to discontinuation of treatment, withdrawal from trial or death will be presented by patient group (Groups A, B and C separately and Groups A and C together) and overall. Data will also be presented by trial part (main and extension) and overall, as applicable. Data will be presented for the safety analysis set, the full exposure safety analysis set and the continuous full exposure safety analysis set.

To quantify the uncertainty in the treatment comparison between Groups A and B for SAEs and TEAEs of special interest in the main part of the trial, a Cox proportional hazards model will be used for time to the first SAE or first TEAE of special interest. This is a descriptive approach; no specific safety hypothesis will be pre-specified, and p-values will generally not be presented for safety data.

Local tolerability results for erythema, swelling and pain scores will be presented descriptively for the main part of the trial.

### Efficacy Analyses

The proportion of patients with mean IGF-1 levels≤1×ULN/<1.3×ULN at Week 50 and Week 52 (average of the 2 measurements) in Groups A and B will be analyzed by a weighted common risk difference test, stratified by prior treatment (octreotide LAR or lanreotide ATG). The ULN derived from the patient's sex and age at screening will be used for the assessment of the individual patient's response. The proportions (95% CI) of patients with mean IGF-1 levels≤1×ULN and mean IGF-1 levels<1.3×ULN at Week 50 and Week 52 in Group C will be presented separately.

Serum levels of IGF-1 will be assessed at the many time points. Pre-dose blood samplings for evaluation of IGF-1 efficacy endpoints in the main part of the trial will be done immediately before CAM2029 administration. In addition, post-dose samples will be taken for a separate evaluation of the PK/PD relationship in the main part of the trial. The date and exact time of IGF-1 sampling must be recorded in the eCRF in both parts of the trial. The IGF-1 samples will be analyzed by a central laboratory. Instructions for sampling and shipping of IGF-1 samples will be provided separately.

Note: wherever normal range or ULN for IGF-1 is mentioned in the document (e.g., IGF-1≤1×ULN), it refers to age- and sex-adjusted values.

The proportions of patients with mean GH cycle levels<2.5 μg/L and <5.0 μg/L at Week 52 and the proportion of patients with mean IGF-1 levels≤1×ULN at Week 50 and Week 52 and mean GH cycle levels<2.5 μg/L at Week 52 will be analyzed and presented in a similar way as the analyses based on IGF-1 levels.

Data for IGF-1 and GH levels over time (absolute values and change from baseline) will be presented for each group and overall.

Time to loss of response (i.e., time to IGF-1 levels>1×ULN and time to IGF-1 levels≥1.3×ULN) will be presented for each group. Only patients who had IGF-1 levels≤1×ULN at baseline will be included in the analysis.

The proportion of patients who are retained in treatment at Week 52 will be presented for each group.

Tumor size data and acromegaly signs and symptoms will be summarized and presented by time point.

Descriptive statistics will be used to summarize the PRO scores over time. Additionally, change from baseline in the AcroQOL and EQ-5D-5L scores in the main part of the trial will be summarized. Data for SF-36 and WPAI in the extension part of the trial will be summarized over time.

Patient satisfaction scale scores in the main part of the trial will be summarized descriptively.

Results from the SIAQ in the main part of the trial will be summarized and change in domain scores will be presented.

### Pharmacokinetic Analyses

The plasma concentrations of octreotide in the main part of the trial will be summarized using descriptive statistics over time by dose, if applicable. A population PK model will be developed using data generated from the current trial together with data from other clinical trials for population PK assessment. The population PK model will be reported separately.

### Exposure-Response Analyses

PK, PD (IGF-1 and GH) and safety (ECG) data generated from this trial may be used together with PK, PD and safety data from other clinical trials for population PK and PK/PD assessments. These data will be reported separately.

### Exploratory and Other Analyses

Prolactin levels (both parts of the trial) and anti-octreotide antibody data (main part of the trial) will be presented descriptively.

Proportion of patients/partners declared competent by the healthcare professional to self- or partner-administer CAM2029 out of those trying will be presented for the main part of the trial.

### Interim Analysis

An interim analysis will be performed when an adequate number of patients have been exposed to CAM2029 for at least 52 weeks. For this analysis, all data available up to the data cut-off will be used.

### EQUIVALENTS

While specific embodiments of the subject disclosure have been discussed, the above specification is illustrative and not restrictive. Many variations of the disclosure will become apparent to those skilled

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A method of treating acromegaly, comprising administering to a patient in need thereof a lipid composition comprising 20 mg of octreotide or a pharmaceutically acceptable salt thereof, wherein the lipid composition is administered to the patient once every 28+/−7 days (once every 4 weeks+/−1 week), and wherein the lipid composition contains about 20 mg octreotide base/mL, about 6.5% w/w anhydrous ethanol, about 6.5% w/w propylene glycol, about 0.01% w/w ethylenediaminetetraacetic acid (EDTA), about 0.01% w/w ethanolamine, and soybean phosphatidylcholine/glycerol dioleate in a weight ratio of about 50/50 to final volume.

2. The method of claim 1, wherein the lipid composition is administered as a unit dose.

3. (canceled)

4. The method of claim 1, wherein the pharmaceutically acceptable salt thereof is hydrochloride.

5. The method of claim 4, wherein the octreotide is the sole active agent in the lipid composition.

6. (canceled)

7. (canceled)

8. (canceled)

9. The method of claim 1, wherein the lipid composition further comprises diethanolamine.

10. The method of claim 1, wherein at least 85 wt. % of the lipid composition consists of octreotide chloride, glycerol dioleate, phosphatidylcholine, ethanol, and propylene glycol.

11. The method of claim 1, wherein at least 86 wt. % of the lipid composition consists of octreotide chloride, glycerol dioleate, phosphatidylcholine, ethanol, propylene glycol, and EDTA, and wherein the lipid composition has a water content of less than 1.0 wt. % at the release of the product for sale.

12. (canceled)

13. The method of claim 1, wherein the volume is about 1 mL.

14. The method of claim 1, comprising administering the lipid composition by a syringe, a pre-filled syringe, an autoinjector, or a pen injector.

15. The method of claim 1, comprising administering the lipid composition by subcutaneous injection.

16. The method of claim 15, wherein the administration provides a depot in the subcutaneous tissue of the patient.

17. The method of claim 1, wherein the patient has an insulin-like growth factor-1 (IGF-1)≤ULN (Upper limit of normal) at week 24 and/or week 52 weeks, after the administration of the lipid composition to the patient.

18. The method of claim 1, wherein the patient has a mean growth hormone (GH) cycle<2.5 ug/L at week 24 and/or week 52, after the administration of the lipid composition to the patient.

19. The method of claim 1, wherein the patient has an improved quality of life (QoL) compared to placebo, as measured by the Acromegaly Quality of Life Questionnaire (ACROQOL) and/or EuroQoL 5D-5L VAS.

20-41. (canceled)

42. The method of claim 1, wherein the method provides improved biochemical and/or symptom control versus standard of care, and/or improved quality of life of patients.

43. (canceled)

44. The method of claim 1, wherein the method provides reduced acromegaly symptom burden as measured by proportion of patients with any acromegaly symptom and the Acromegaly Index of Severity (AIS) score.

45. The method of claim 44, wherein the AIS score is the sum of scores of the six acromegaly symptoms of headache, sweating, fatigue, joint pain, paresthesia, and soft tissue swelling.

46. The method of claim 1, wherein the lipid composition is substantially non-aqueous.

47. The method of claim 1, wherein the lipid composition has an initial absolute level of water between 0 to 1.0 wt. %.

48. The method of claim 1, wherein the lipid composition has no more than 1.5 wt. % water after 3 months of storage.

49. The method of claim 1, wherein the patient has a maximum blood plasma concentration (Cmax) of octreotide at steady state of less than 45 ng/ml for each administration.

50. The method of claim 1, wherein the patient has an average blood plasma concentration (C_{AV}) of octreotide at steady state of 3-24 ng/ml for each administration.

51. The method of claim 1, wherein the patient has a plasma concentration AUC of octreotide at steady state of 1300-6700 ng*h/ml for each administration.

52. The method of claim 1, wherein the patient has a maximum blood plasma concentration (Cmax) of octreotide at steady state of less than 45 ng/ml after each administration of the lipid composition.

53. The method of claim 1, wherein the patient has an average blood plasma concentration (C_{AV}) of octreotide at steady state of 6-40 ng/ml after each administration of the lipid composition.

54. The method of claim 1, wherein the patient has a plasma concentration AUC of octreotide at steady state of 1000-6700 ng*h/ml after each administration of the lipid composition.

55. The method of claim 1, wherein administration of the lipid composition provides an improved IGF-1 response rate over 52 weeks.
